(19) United States
(12) Patent Application Publication
(10) Pub. No.: US 2012/0115724 A1
(43) Pub. Date: May 10, 2012

(54) METHOD OF CONTROLLING UNDESIRED VEGETATION

(75) Inventors: William Guy Whittingham, Berkshire (GB); Harry Glithro, Berkshire (GB); Caroline Louise Winn, Berkshire (GB); Mary Bernadette Aspinall, Berkshire (GB); Claudio Screpanti, Berkshire (GB)

(73) Assignee: SYNGENTA CROP PROTECTION LLC, Greensboro, NC (US)

(21) Appl. No.: 13/318,387

(22) PCT Filed: Apr. 27, 2010
(86) PCT No.: PCT/GB2010/000823

§ 371 Date: Jan. 30, 2012

(30) Foreign Application Priority Data
May 1, 2009 (GB) 0907625.8

(51) Int. Cl. A01N 57/20; C07D 239/42; C07D 405/12; C07D 401/12; A01P 21/00; A01P 7/04; A01P 7/02; A01P 5/00; A01P 9/00; A01P 3/00; A01N 43/54; A01P 13/00

(52) U.S. Cl. 504/128; 504/239; 504/136; 544/329; 544/328

(57) ABSTRACT

The present invention relates to methods of controlling undesired plant growth in crops of soya through the use of certain substituted pyrimidine derivatives. It also relates to certain novel substituted pyrimidine derivatives. Effective weed control is thereby obtained, whilst at the same time achieving unexpected levels of crop safety.

The present invention relates to methods of controlling undesired plant growth in crops of soya, through the use of certain substituted pyrimidine derivatives. It also relates to certain novel substituted pyrimidine derivatives. Effective weed control is thereby obtained, whilst at the same time achieving unexpected levels of crop safety.

Herbicidal substituted pyrimidine derivatives are known in the prior art. For example pyrimidine derivatives comprising an optionally substituted cyclopropyl or optionally substituted phenyl group at position 2 in combination with inter alia a nitro or optionally substituted amino group at the position 6 of the pyrimidine ring and their use as herbicides are disclosed in International Patent Publication No. WO 2005/063721. International Patent Publication No. WO 2007/082076 discloses a number of 2-(poly-substituted aryl)-6-amino-5-halo-4-pyrimidine carboxylic acids and their use as herbicides, whilst International Patent Publication No. WO 2007/092184 discloses certain substituted pyrimidine carboxylic acid derivatives as compounds capable of improving the harvestability of crops. WO 2009/023438 describes 2-(2-fluoro-substituted-phenyl)-6-amino-5-chloro-4-pyrimidine carboxylates and their use as herbicidal compounds in corn. WO 2009/046090 describes 2-substituted-6-amino-5-alkyl/alkenyl/alkynyl-4-pyrimidinecarboxylic acid derivatives and their use as herbicides in turf and ornamental environments as well as in crops of corn, rice and cereals. WO 2009/081112 discloses certain 6-amino pyrimidine derivatives, wherein the amino group is substituted with an optionally substituted 3-8 membered ring and their use as herbicides. WO 2009/138712 discloses certain 6-amino pyrimidine derivatives, with a heteroaromatic group at the 2-position of the pyrimidine ring and their use as herbicides. However, it can be seen that compounds of the prior art demonstrate not only herbicidal activity with respect to undesired plant growth, but may also exhibit damage to crop plants.

The present invention is based on the surprising finding that certain substituted pyrimidinecarboxylic acid/ester derivatives, in particular those wherein the combination of an optionally substituted phenyl or pyridyl moiety at the 2-position with an optionally substituted alkenyl moiety at position 5 of the pyrimidine ring, exhibit not only herbicidal activity but also an unexpected level of crop safety in crops of soya.

Thus in a first aspect the invention provides a method of controlling undesired plant growth in a crop of soya plants which comprises applying to said undesired plants or to the locus of said undesired plants, or to said soya plants, a compound of formula (I)

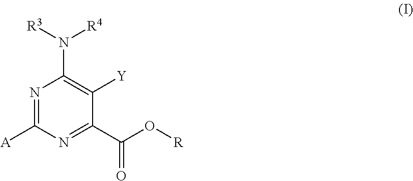

or salt or N-oxide thereof, wherein: A is phenyl optionally substituted by 1-4 groups R¹, or pyridyl optionally substituted by 1-4 groups R¹;each R¹ is independently: halogen; cyano; nitro; alkyl; haloalkyl; alkoxyalkyl; alkoxy; haloalkoxy; alkylthio, alkylsulphinyl, alkylsulphonyl, alkylcarbonyl, alkoxycarbonyl; amino, alkylamino, dialkylamino; R³ is hydrogen, C₁₋₄ alkyl, SO₂R⁶, or C(O)R⁷; R⁴ is hydrogen, C₁₋₄ alkyl optionally substituted by 1-3 groups R⁵, or C₃-₆ cycloalkyl; each R⁵ is independently: hydroxyl; cycloalkyl; phenyl optionally substituted by 1-3 groups R⁹; heteroaryl optionally substituted by 1-3 groups R¹⁰; each R⁶ is independently C₁₋₄ alkyl or phenyl; each R⁷ is independently C₁-₄ alkyl, phenyl, or C₁₋₄alkoxy; each R⁹ is independently: halogen; cyano; nitro; alkyl; haloalkyl; alkoxy; haloalkoxy; alkoxycarbonyl; amino; alkylamino; or dialkylamino; each R¹⁰ is independently: halogen; cyano; alkyl; haloalkyl; alkoxy; haloalkoxy; alkoxycarbonyl; amino; alkylamino; or dialkylamino; Y is C₂₋₄alkenyl optionally substituted by 1-3 groups R¹⁴; each R¹⁴ is independently halogen, cyano, cycloalkyl, alkylcarbonyl, alkoxycarbonyl, alkoxy, alkylthio, alkylsulphinyl, alkylsulphonyl; and R is hydrogen, C₁₋₁₀ alkyl, C₁₋₄alkoxyC₂₋₄alkyl, C₃₋₁₀ alkenyl or phenylC₁₋₂alkyl.

As described above, the method of the invention involves applying to undesired plants or to the locus thereof, or to a crop of soya plants, a herbicidally effective amount of a compound of formula (I). The invention also extends to the use of a composition (e.g. formulation) or mixture as described hereinafter, said composition or mixture comprising a compound of formula (I). The invention thus also relates to a method of inhibiting undesired plant growth which comprises applying to the undesired plants or to the locus thereof a herbicidally effective amount of a compound of formula (I), composition, or mixture as described herein.

Any method of application to the undesired plants/soya plants, or locus thereof, which is routinely used in agriculture may be used, for example application by spray or broadcast method typically after suitable dilution of a compound of formula (I) (whether said compound is formulated and/or in combination with one or more further active ingredients as described herein).

Methods of the invention may employ a pre-emergence application and/or a post-emergence application of a compound of formula (I). It is particularly preferred that compounds of formula (I) are employed post-emergence.

The rates of application of compounds of formula (I) may vary within wide limits and depend on the nature of the soil, the method of application (pre- or post-emergence; seed dressing; application to the seed furrow; no tillage application etc.), the soya crop, or weed to be controlled, the prevailing climatic conditions, and other factors governed by the method of application, the time of application, and the soya crop. The compounds of formula I according to the invention are generally applied at a rate of from 5 to 2000 g/ha, preferably from 25 to 1000 g/ha, and more preferably still at a rate of from 25 to 250 g/ha.

The term “locus” as used herein includes not only areas where weeds may already be growing, but also areas where weeds have yet to emerge, and also to areas under cultivation with respect to crops of soya plants. Areas under cultivation include land on which soya plants are already growing and land intended for cultivation with such a soya crop.

The terms “undesired plant growth” and “undesired plants” refers not only to agronomically important weeds as described below, but also to volunteer crop plants.

Methods of the invention may be used to control a large number of agronomically important weeds. The weeds that may be controlled include both monocotyledonous and dicotyledonous weeds, such as, for example, Alisma spp, Leptochloa, Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum, Rottboellia, Cyperus and especially Cyperus iria, Abutilon, Sida, Xanthium, Amaranthus, Chenopodium, Ipomoea, Bidens, Euphorbia, Chrysanthemum, Galium, Viola, Veronica, Ischaemum, Polygonum, Helianthus, Panicum, Eriochloa, Brachiaria, Cenchrus, Commelina, Spermacoce, Senna, Tridax, Richardia, Chamaesyce, and Conyza spp. In particular, weeds that are controlled according to methods of the invention are those prevalent in areas where soya crops are grown. Thus, it is particularly preferred that methods of the invention are used to control weeds of the following species: Euphorbia, Bidens, Ipomoea, Sida, Commelina, Conyza, Polygonum, Helianthus, Panicum, Eriochloa, Brachiaria, Cenchrus, Sorghum, and Scirpus.

Method of the invention may be employed in crops of soya which have been rendered tolerant to herbicides or classes of herbicides (e.g. ALS-, GS-, and EPSPS-inhibitors) by conventional methods of breeding or by genetic engineering. Examples of soybeans that have been rendered tolerant to herbicides by genetic engineering methods include e.g. glyphosate- and glufosinate-resistant soya varieties commercially available under the trade names RoundupReady® and LibertyLink® as well as soybean that has been engineered to be resistant to dicamba, phenoxypropionic acids, pyridyloxyacetic acids and/or picolinate auxines.

Crops are also to be understood as being those which have been rendered resistant to harmful insects and fungi by genetic engineering methods. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

The compounds of formula (I) that are employed in the methods for the invention may exist in different geometric or optical isomers or different tautomeric forms. One or more centres of chirality may be present, in which case compounds of the formula (I) may be present as pure enantiomers, mixtures of enantiomers, pure diastereomers or mixtures of diastereomers. There may be double bonds present in the molecule, such as C═C or C═N bonds, in which case compounds of formula (I) may exist as single isomers or mixtures of isomers. Centres of tautomerisation may be present. This invention covers all such isomers and tautomers and mixtures thereof in all proportions as well as isotopic forms such as deuterated compounds.

For the avoidance of doubt, the term “compound” as used herein includes all salts and N-oxides of said compound.

Suitable salts include those formed by contact with acids or bases. Suitable acid addition salts include those with an inorganic acid such as hydrochloric, hydrobromic, sulfuric, nitric and phosphoric acids, or an organic carboxylic acid such as oxalic, tartaric, lactic, butyric, toluic, hexanoic and phthalic acids, or sulphonic acids such as methane, benzene and toluene sulphonic acids. Other examples of organic carboxylic acids include haloacids such as trifluoroacetic acid.

Suitable salts also include those formed by strong bases (e.g. metal hydroxides - in particular sodium, potassium or lithium- or quaternary ammonium hydroxide) as well as those formed with amines.

N-oxides are oxidised forms of tertiary amines or oxidised forms of nitrogen containing heteroaromatic compounds. They are described in many books for example in “Heterocyclic N-oxides” by Angelo Albini and Silvio Pietra, CRC Press, Boca Raton, Fla., 1991.

Each alkyl moiety either alone or as part of a larger group (such as alkoxyalkyl, alkylthio, alkylsulphinyl, alkylsulphonyl, alkylcarbonyl, alkoxycarbonyl etc.) is a straight or branched chain and is, for example, methyl, ethyl, n-propyl, n-butyl, n-pentyl, n-hexyl, iso-propyl, sec-butyl, iso-butyl, tert-butyl or neo-pentyl. The alkyl groups are suitably C₁ to C₁₀ alkyl groups, but are preferably C₁-C₈, even more preferably C₁-C₆ and most preferably C₁-C₄ alkyl groups.

Alkenyl moieties can be in the form of straight or branched chains, and where appropriate, can be of either the (E)- or (Z)-configuration. Examples are vinyl and allyl. Alkenyl moieties can contain one or more double bonds in any combination.

Halogen is fluorine, chlorine, bromine or iodine.

Haloalkyl groups are alkyl groups which are substituted with one or more of the same or different halogen atoms and are, for example, CF₃, CF₂Cl, CF₂H, CCl₂H, ClCH₂, BrCH₂, CH₃CHF, (CH₃)₂CF, CF₃CH₂ or CHF₂CH₂.

The terms “heteroaryl”, “heteroaromatic ring” or “heteroaromatic ring system” refer to an aromatic ring system containing at least one heteroatom and consisting either of a single ring or of two or more fused rings. Preferably, single rings will contain up to three and bicyclic systems up to four heteroatoms which will preferably be chosen from nitrogen, oxygen and sulphur. Examples of such groups include furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzisofuryl, benzothienyl, benzisothienyl, indolyl, isoindolyl, indazolyl, benzothiazolyl, benzisothiazolyl, benzoxazolyl, benzisoxazolyl, benzimidazolyl, 2,1,3-benzoxadiazole, quinolinyl, isoquinolinyl, cinnolinyl, phthalazinyl, quinazolinyl, quinoxalinyl, naphthyridinyl, benzotriazinyl, purinyl, pteridinyl and indolizinyl. Preferred examples of heteroaromatic radicals include pyridyl, pyrimidyl, triazinyl, thienyl, furyl, oxazolyl, isoxazolyl, 2,1,3-benzoxadiazole and thiazolyl.

In the case of heteroaromatic rings containing S as a heteroatom, the S atom may also be in the form of a mono- or di-oxide.

Cycloalkyl includes cyclopropyl, cyclobutyl, cyclopentyl and cyclohexyl. Cycloalkylalkyl is preferentially cyclopropylmethyl.

For substituted phenyl moieties and heteroaryl groups it is preferred that one or more substituents are independently selected from halogen, C₁₋₆ alkyl, C₁₋₆ haloalkyl, C₁₋₆ alkoxy(C₁₋₆)alkyl, C₁₋₆ alkoxy, C₁₋₆ haloalkoxy, C₁₋₆ alkylthio, C₁₋₆ haloalkylthio, C₁₋₆ alkylsulfinyl, C₁₋₆ haloalkylsulfinyl, C₁₋₆ alkylsulfonyl, C₁₋₆ haloalkylsulfonyl, C₂₋₆ alkenyl, C₂₋₆ haloalkenyl, C₂₋₆ alkynyl, C₃₋₇ cycloalkyl, nitro, cyano, CO₂H, C₁₋₆ alkylcarbonyl, C₁₋₆ alkoxycarbonyl, aryl, heteroaryl, C₁₋₆ alkylamino, di(C₁₋₆ alkyl)amino, C₁₋₆ alkylaminocarbonyl, or di(C₁₋₆ alkyl)aminocarbonyl.

Haloalkenyl groups are alkenyl groups which are substituted with one or more of the same or different halogen atoms.

It is to be understood that dialkylamino substituents include those where the dialkyl groups together with the N atom to which they are attached form a five, six or seven-membered heterocyclic ring which may contain one or two further heteroatoms selected from O, N or S and which is optionally substituted by one or two independently selected (C₁₋₆)alkyl groups. When heterocyclic rings are formed by joining two groups on an N atom, the resulting rings are suitably pyrrolidine, piperidine, thiomorpholine and morpholine each of which may be substituted by one or two independently selected (C₁₋₆) alkyl groups.

In particularly preferred embodiments of the invention, the preferred groups for A, R¹, R³, R⁴, R⁵, R⁹, R¹⁰, Y, R, and Z, in any combination thereof, are as set out below.

Preferably, the ring A is a phenyl group optionally substituted by 1-3 groups R¹, or a pyridyl group optionally substituted by 1-3 groups R¹. In the more preferred embodiments, each R¹ is independently: halogen; C₁₋₂alkyl; haloC₁₋₂alkyl; C₁₋₂alkoxyC₁₋₂alkyl; C₁₋₂alkoxy; haloC₁₋₂alkoxy; C₁₋₂alkylthio; amino, C₁₋₂alkylamino, di-C₁₋₂alkylamino;

More preferably still, A is a group of formula (II)

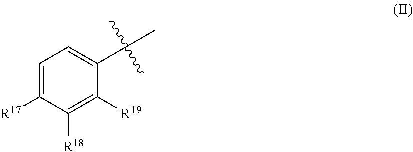

wherein R¹⁷ is methyl, or halogen; R¹⁸ is H, F, Cl, C₁₋₂alkyl, C₁₋₂haloalkyl, OR²⁰, or N(R²⁰)₂; R¹⁹ is H, F, or Cl; and each R²⁰ is independently H, C₁₋₂alkyl, C₁₋₂haloalkyl.

Preferably R³ is hydrogen, or C₁₋₂ alkyl. Most preferably R³ is hydrogen.

Preferably R⁴ is hydrogen, or C₁₋₂ alkyl optionally substituted by one or more R⁵.

Preferably each R⁵ is independently, phenyl optionally substituted by 1-2 groups R⁹; furanyl optionally substituted by 1-2 groups R¹⁰; or pyridyl optionally substituted by 1-2 groups R¹⁰.

Most preferably R⁴ is hydrogen, 2-nitrophenyl-methyl, or furanyl-methyl.

Preferably each R⁹ is independently: halogen; cyano; nitro; C₁₋₂alkyl; C₁₋₂haloalkyl; C₁₋₂alkoxy; C₁₋₂haloalkoxy; amino; C₁₋₂alkylamino; or diC₁₋₂alkylamino.

Preferably each R¹⁹ is independently: halogen; cyano; C₁₋₂alkyl; C₁₋₂haloalkyl; C₁₋₂alkoxy; C₁₋₂haloalkoxy; amino; C₁₋₂alkylamino; or di-C₁₋₂alkylamino.

Preferably Y is C₂₋₄alkenyl or C₂₋₄haloalkenyl, more preferably C₂₋₃alkenyl.

Preferably R is hydrogen, C₁₋₈ alkyl, C₁₋₄ alkoxyethyl, allyl or phenylmethyl, more preferably hydrogen or C₁₋₄alkyl.

Preferred compounds for use in the invention include compounds numbered 1 to 48 as listed in the Examples. Particularly preferred compounds for use in the invention include compounds numbered 1, 2, 3, 4, 5, 6, 9, 10, 11, 12, 13, 15, 16, 26, 34, 35, 36, 38, 39, 41, 42, 44, 45, 46, 47 and 48 as listed in the Examples.

In a further aspect, the invention provides a compound of formula (I):

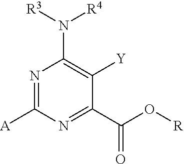

wherein A is

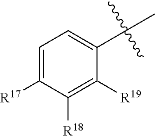

R¹⁷ is methyl, or halogen; R¹⁸ is H, F, Cl, C₁₋₂alkyl, C₁₋₂haloalkyl, or N(R²⁰)₂; R¹⁹ is H, F, or Cl; each R²⁰ is independently H, C₁₋₂alkyl, C₁₋₂haloalkyl; provided that both R¹⁸ and R¹⁹ are not hydrogen; R³ is hydrogen, C₁₋₄alkyl, SO₂R⁶, or C(O)R⁷; R⁴ is C₁₋₄alkyl substituted by 1-3 groups R⁵ or C cycloalkyl; each R⁵ is, independently, hydroxyl, cycloalkyl, phenyl optionally substituted by 1-3 groups R⁹, heteroaryl optionally substituted by 1-3 groups R¹⁰; each R⁶ is independently C₁₋₄alkyl or phenyl; each R⁷ is independently C₁₋₄ alkyl, phenyl, or C₁₋₄ alkoxy; each R⁹ is, independently, halogen, cyano, nitro, alkyl, haloalkyl, alkoxy, haloalkoxy, alkoxycarbonyl, amino, alkylamino or dialkylamino; each R¹⁰ is, independently, halogen, cyano, alkyl, haloalkyl, alkoxy, haloalkoxy, alkoxycarbonyl, amino, alkylamino or dialkylamino; Y is C₂₋₄alkenyl optionally substituted by 1-3 groups R¹⁴; each R¹⁴ is, independently, halogen, cyano, cycloalkyl, alkylcarbonyl, alkoxycarbonyl, alkoxy, alkylthio, alkylsulphinyl or alkylsulphonyl; and R is hydrogen, C₁₋₁₀ alkyl, C₁₋₄alkoxyC₂₋₄alkyl, C₃₋₁₀ alkenyl or phenylC₁₋₂alkyl.

Preferred compounds of the invention include compounds numbered 13, 16, 17, 18, 19, 20, 21, 22 and 23, as listed in the Examples.

These compounds may be made as described herein in the Examples, or, as the skilled man will appreciate by applying and/or adapting as appropriate, the methods described in the prior art (see for example WO 2009/046090, WO2009/081112 and WO2009/138712).

Compounds of formula (I) may be used in methods of the invention in unmodified form, i.e. as obtainable from synthesis, but preferably are formulated in any suitable manner using formulation adjuvants, such as carriers, solvents and surface-active substances, for example, as described hereinafter. Thus in a further aspect methods of the invention will employ a compound of formula (I) formulated with at least one agriculturally acceptable formulation adjuvant or diluent.

The formulations can be in various physical forms, e.g. in the form of dusting powders, gels, wettable powders, water-dispersible granules, water-dispersible tablets, effervescent pellets, emulsifiable concentrates, microemulsifiable concentrates, suspension concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO Specifications for Plant Protection Products, 5th Edition, 1999. The formulations can be in the form of concentrates which are diluted prior to use, although ready-to-use formulations can also be made. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof. The active ingredients can also be contained in very fine microcapsules consisting of a polymer. Microcapsules usually have a diameter of from 0.1 to 500 microns. Typically, they will contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other known polymers. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of compositions for use in the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octa-decanoic acid, octylamine acetate, oleic acid, oleylamine, o-xylene, phenol, polyethylene glycol (PEG), propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like. Water is generally the carrier of choice for diluting the concentrates. Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances, as described, for example, in 40 CFR 180.910 and 40 CFR180.920.

A large number of surface-active substances may advantageously be used in the formulations, especially in those formulations designed to be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in “McCutcheon's Detergents and Emulsifiers Annual” MC Publishing Corp., Ridgewood N.J., 1981.

Further adjuvants that can usually be used in the formulations described herein include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and also liquid and solid fertilisers.

Compositions for use in methods of the invention can additionally include an additive comprising an oil of vegetable or animal origin, a mineral oil, alkyl esters of such oils or mixtures of such oils and oil derivatives. The amount of oil additive in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil, olive oil or sunflower oil, emulsified vegetable oil, such as AMIGO® (Rhône-Poulenc Canada Inc.), alkyl esters of oils of vegetable origin, for example the methyl derivatives, or an oil of animal origin, such as fish oil or beef tallow: A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈₋₂₂ fatty acids, especially the methyl derivatives of C₁₂₋₁₈ fatty acids, for example the methyl esters of lauric acid, palmitic acid and oleic acid, being of importance. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000. Another preferred adjuvant is Adigor® (Syngenta AG) which is a methylated rapeseed oil-based adjuvant.

The application and action of the oil additives can be further improved by combination with surface-active substances, such as non-ionic, anionic or cationic surfactants. Examples of suitable anionic, non-ionic and cationic surfactants are listed on pages 7 and 8 of WO97/34485. Preferred surface-active substances are anionic surfactants of the dodecylbenzylsulfonate type, especially the calcium salts thereof, and also non-ionic surfactants of the fatty alcohol ethoxylate type. Special preference is given to ethoxylated C₁₂₋₂₂ fatty alcohols having a degree of ethoxylation of from 5 to 40. Examples of commercially available surfactants are the Genapol types (Clariant AG). Also preferred are silicone surfactants, especially polyalkyl-oxide-modified heptamethyltriloxanes which are commercially available e.g. as Silwet L-77®, and also perfluorinated surfactants. The concentration of the surface-active substances in relation to the total additive is generally from 1 to 30% by weight. Examples of oil additives consisting of mixtures of oil or mineral oils or derivatives thereof with surfactants are Edenor ME SU®, Turbocharge® (Syngenta AG, CH) or ActipronC (BP Oil UK Limited, GB).

If desired, it is also possible for the mentioned surface-active substances to be used in the formulations on their own, that is to say, without oil additives.

Furthermore, the addition of an organic solvent to the oil additive/surfactant mixture may contribute to an additional enhancement of action. Suitable solvents are, for example, Solvesso® (ESSO) or Aromatic Solvent® (Exxon Corporation). The concentration of such solvents can be from 10 to 80% by weight of the total weight. Oil additives that are present in admixture with solvents are described, for example, in U.S. Pat. No. 4,834,908. A commercially available oil additive disclosed therein is known by the name MERGE® (BASF Corporation). A further oil additive that is preferred according to the invention is SCORE®(Syngenta Crop Protection Canada).

In addition to the oil additives listed above, for the purpose of enhancing the action of the compositions according to the invention it is also possible for formulations of alkylpyrrolidones (e.g. Agrimax®) to be added to the spray mixture. Formulations of synthetic lattices, e.g. polyacrylamide, polyvinyl compounds or poly-1-p-menthene (e.g. Bond®, Courier® or Emerald®) may also be used. It is also possible for solutions that contain propionic acid, for example Eurogkem Pen-e-trate®, to be added to the spray mixture as action-enhancing agent.

Herbicidal compositions/formulations for use in the invention generally comprise from 0.1 to 99% by weight, especially from 0.1 to 95% by weight, compounds of formula (I) and from 1 to 99.9% by weight of a formulation adjuvant which preferably includes from 0 to 25% by weight of a surface-active substance. Whereas commercial products will preferably be formulated as concentrates, the end user will normally employ dilute formulations.

Examples of preferred formulation types and their typical compositions are given below (% is percent by weight). Suspension concentrates and emulsifiable concentrates as described herein are particularly preferred types of formulation for use in the invention.

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%
   The following Examples further illustrate, but do not limit, the invention.
   Formulation Examples for Herbicides of Formula (I) (%=% b weight)

F1. Emulsifiable concentrates

| | a) | b) | c) | d) |
|---|---|---|---|---|
| active ingredient | 5% | 10% | 25% | 50% |
| calcium dodecylbenzenesulfonate | 6% | 8% | 6% | 8% |
| castor oil polyglycol ether | 4% | — | 4% | 4% |
| (36 mol of ethylene oxide) | | | | |
| octylphenol polyglycol ether | — | 4% | — | 2% |
| (7-8 mol of ethylene oxide) | | | | |
| NMP | — | — | 10% | 20% |
| arom. hydrocarbon mixture | 85% | 78% | 55% | 16% |
| (C₉-C₁₂) | | | | |

Emulsions of any desired concentration can be obtained from such concentrates by dilution with water.

F2. Solutions

| | a) | b) | c) | d) |
|---|---|---|---|---|
| active ingredient | 5% | 10% | 50% | 90% |
| 1-methoxy-3-(3-methoxy- | — | 20% | 20% | — |
| propoxy)-propane | | | | |
| polyethylene glycol MW 400 | 20% | 10% | — | — |
| NMP | — | — | 30% | 10% |
| arom. hydrocarbon mixture | 75% | 60% | — | — |
| (C₉-C₁₂) | | | | |

The solutions are suitable for use in the form of microdrops.

F3. Wettable powders

| | a) | b) | c) | d) |
|---|---|---|---|---|
| active ingredient | 5% | 25% | 50% | 80% |
| sodium lignosulfonate | 4% | — | 3% | — |
| sodium lauryl sulfate | 2% | 3% | — | 4% |
| sodium diisobutylnaphthalene- | — | 6% | 5% | 6% |
| sulfonate | | | | |
| octylphenol polyglycol ether | — | 1% | 2% | — |
| (7-8 mol of ethylene oxide) | | | | |
| highly dispersed silicic acid | 1% | 3% | 5% | 10% |
| kaolin | 88% | 62% | 35% | — |

The active ingredient is mixed thoroughly with the adjuvants and the mixture is thoroughly ground in a suitable mill, affording wettable powders which can be diluted with water to give suspensions of any desired concentration.

F4. Coated granules

| | a) | b) | c) |
|---|---|---|---|
| active ingredient | 0.1% | 5% | 15% |
| highly dispersed silicic acid | 0.9% | 2% | 2% |
| inorganic carrier | 99.0% | 93% | 83% |
| (diameter 0.1-1 mm) | | | |
| e.g., CaCO₃ or SiO₂ | | | |

The active ingredient is dissolved in methylene chloride and applied to the carrier by spraying, and the solvent is then evaporated off in vacuo.

F5. Coated granules

| | a) | b) | c) |
|---|---|---|---|
| active ingredient | 0.1% | 5% | 15% |
| polyethylene glycol MW 200 | 1.0% | 2% | 3% |
| highly dispersed silicic acid | 0.9% | 1% | 2% |
| inorganic carrier | 98.0% | 92% | 80% |
| (diameter 0.1-1 mm) | | | |
| e.g., CaCO₃ or SiO₂ | | | |

The finely ground active ingredient is uniformly applied, in a mixer, to the carrier moistened with polyethylene glycol. Non-dusty coated granules are obtained in this manner.

F6. Extruder granules

| | a) | b) | c) | d) |
|---|---|---|---|---|
| active ingredient | 0.1% | 3% | 5% | 15% |
| sodium lignosulfonate | 1.5% | 2% | 3% | 4% |
| carboxymethylcellulose | 1.4% | 2% | 2% | 2% |
| kaolin | 97.0% | 93% | 90% | 79% |

The active ingredient is mixed and ground with the adjuvants, and the mixture is moistened with water. The mixture is extruded and then dried in a stream of air.

F7. Dusts

| | a) | b) | c) |
|---|---|---|---|
| active ingredient | 0.1% | 1% | 5% |
| talcum | 39.9% | 49% | 35% |
| kaolin | 60.0% | 50% | 60% |

Ready-to-use dusts are obtained by mixing the active ingredient with the carriers and grinding the mixture in a suitable mill.

F8. Suspension concentrates

| | a) | b) | c) | d) |
|---|---|---|---|---|
| active ingredient | 3% | 10% | 25% | 50% |
| ethylene glycol | 5% | 5% | 5% | 5% |
| nonylphenol polyglycol ether | — | 1% | 2% | — |
| (15 mol of ethylene oxide) | | | | |
| sodium lignosulfonate | 3% | 3% | 4% | 5% |
| carboxymethylcellulose | 1% | 1% | 1% | 1% |
| 37% aqueous formaldehyde | 0.2% | 0.2% | 0.2% | 0.2% |
| solution | | | | |
| silicone oil emulsion | 0.8% | 0.8% | 0.8% | 0.8% |
| water | 87% | 79% | 62% | 38% |

The finely ground active ingredient is intimately mixed with the adjuvants, giving a suspension concentrate from which suspensions of any desired concentration can be obtained by dilution with water.

As mentioned above, methods of the invention may employ compounds of formula (I) (formulated or not) in combination with other active ingredients, e.g. other herbicides, and/or insecticides, and/or acaricides, and/or nematocides, and/or molluscicides, and/or fungicides, and/or plant growth regulators. Such mixtures, and the use of such mixtures to control weeds and/or undesired plant growth form yet further aspects of the invention. For the avoidance of doubt, the use of a compound of formula (I) in combination with at least one further active ingredient encompasses simultaneous application of both a compound of formula (I) and the further active ingredient, as well as separate (subsequent) applications of each.

Where a compound of formula (I) is combined with at least one additional herbicide, the following mixtures of the compound of formula (I) are particularly preferred. Compound of formula (I)+acetochlor, compound of formula (I)+acifluorfen, compound of formula (I)+acifluorfen-sodium, compound of formula (I)+alloxydim, compound of formula (I)+aminocyclopyrachlor, compound of formula (I)+ammonium sulfamate, compound of formula (I)+bentazone, compound of formula (I)+bispyribac, compound of formula (I)+bispyribac-sodium, compound of formula (I)+carfentrazone, compound of formula (I)+carfentrazone-ethyl, compound of formula (I)+chlorimuron, compound of formula (I)+chlorimuron-ethyl, compound of formula (I)+chlorsulfuron, compound of formula (I)+clethodim, compound of formula (I)+clodinafop, compound of formula (I)+clodinafop-propargyl, compound of formula (I)+clomazone, compound of formula (I)+cloransulam, compound of formula (I)+cloransulam-methyl, compound of formula (I)+cyhalofop, compound of formula (I)+cyhalofop-butyl, compound of formula (I)+2,4-D, compound of formula (I)+2,4-DB, compound of formula (I)+dicamba, compound of formula (I)+diclosulam, compound of formula (I)+diflufenzopyr, compound of formula (I)+dimethenamid, compound of formula (I)+dimethenamid-P, compound of formula (I)+diquat, compound of formula (I)+diquat dibromide, compound of formula (I)+fenoxaprop, compound of formula (I)+fenoxaprop-P, compound of formula (I)+fenoxaprop-ethyl, compound of formula (I)+fenoxaprop-P-ethyl, compound of formula (I)+fluazifop, compound of formula (I)+fluazifop-butyl, compound of formula (I)+fluazifop-P, compound of formula (I)+fluazifop-P-butyl, compound of formula (I)+flufenacet, compound of formula (I)+flumiclorac, compound of formula (I)+flumiclorac-pentyl, compound of formula (I)+flumioxazin, compound of formula (I)+fluthiacet, compound of formula (I)+fluthiacet-methyl, compound of formula (I)+fomesafen, compound of formula (I)+glufosinate, compound of formula (I)+glufosinate-ammonium, compound of formula (I)+glyphosate, compound of formula (I)+halosulfuron, compound of formula (I)+halosulfuron-methyl, compound of formula (I)+haloxyfop, compound of formula (I)+haloxyfop-P, compound of formula (I)+imazamethabenz, compound of formula (I)+imazamethabenz-methyl, compound of formula (I)+imazamox, compound of formula (I)+imazapyr, compound of formula (I)+imazaquin, compound of formula (I)+imazethapyr, compound of formula (I)+imazosulfuron, compound of formula (I)+iodosulfuron, compound of formula (I)+iodosulfuron-methyl-sodium, compound of formula (I)+isoxaflutole, compound of formula (I)+lactofen, compound of formula (I)+linuron, compound of formula (I)+mesosulfuron, compound of formula (I)+mesosulfuron-methyl, compound of formula (I)+mesotrione, compound of formula (I)+metolachlor, compound of formula (I)+S-metolachlor, compound of formula (I)+metosulam, compound of formula (I)+metribuzin, compound of formula (I)+metsulfuron, compound of formula (I)+metsulfuron-methyl, compound of formula (I)+NC-620, compound of formula (I)+nicosulfuron, compound of formula (I)+oxasulfuron, compound of formula (I)+paraquat, compound of formula (I)+paraquat dichloride, compound of formula (I)+pendimethalin, compound of formula (I)+penoxsulam, compound of formula (I)+petrolium oils, compound of formula (I)+picloram, compound of formula (I)+pinoxaden, compound of formula (I)+primisulfuron, compound of formula (I)+primisulfuron-methyl, compound of formula (I)+propropyrisulfuron (TH-547), compound of formula (I)+prosulfuron, compound of formula (I)+pyrasulfotole, compound of formula (I)+pyrazosulfuron, compound of formula (I)+pyrazosulfuron-ethyl, compound of formula (I)+pyribenzoxim, compound of formula (I)+pyriftalid, compound of formula (I)+pyriminobac, compound of formula (I)+pyriminobac-methyl, compound of formula (I)+pyrimisulfan, compound of formula (I)+pyrithiobac, compound of formula (I)+pyrithiobac-sodium, compound of formula (I)+pyroxasulfone, formula (I)+pyroxsulam, compound of formula (I)+quinclorac, compound of formula (I)+quizalofop, compound of formula (I)+quizalofop-P, compound of formula (I)+quizalofop-ethyl, compound of formula (I)+quizalofop-P-ehtyl, compound of formula (I)+rimsulfuron, compound of formula (I)+saflufenacil, compound of formula (I)+sethoxydim, compound of formula (I)+sulcotrione, compound of formula (I)+sulfentrazone, compound of formula (I)+sulfometuron, compound of formula (I)+sulfometuron-methyl, compound of formula (I)+sulfosate, compound of formula (I)+sulfosulfuron, compound of formula (I)+tefuryltrione, compound of formula (I)+tembotrione, compound of formula (I)+tepraloxydim, compound of formula (I)+thifensulfuron, compound of formula (I)+thiencarbazone, compound of formula (I)+thifensulfuron-methyl, compound of formula (I)+topramezone, compound of formula (I)+tralkoxydim, compound of formula (I)+triasulfuron, compound of formula (I)+tribenuron, compound of formula (I)+tribenuron-methyl, compound of formula (I)+trifloxysulfuron, compound of formula (I)+trifloxysulfuron-sodium, compound of formula (I)+trifluralin, compound of formula (I)+triflusulfuron, compound of formula (I)+triflusulfuron-methyl, and compound of formula (I)+trinexapac-ethyl.

Whilst two-way mixtures of a compound of formula (I) and another herbicide are explicitly disclosed above, the skilled man will appreciate that the invention extends to three-way, and further multiple combinations comprising the above two-way mixtures.

In preferred embodiments a compound of formula (I) is combined with an acetolactate synthase inhibitor, (e.g. one or more of metsulfuron, thifensulfuron, tribenuron, triasulfuron, iodosulfuron, mesosulfuron, sulfosulfuron and pyroxsulam, as well as salts or esters thereof), a synthetic auxin herbicide (e.g. one or more of aminocyclopyrachlor, 2,4-D, 2,4-DB and dicamba), an ACCase-inhibiting herbicide (e.g. one or more of a phenylpyrazoline herbicide such as pinoxaden; an aryloxyphenoxypropionic herbicide such as clodinafop, cyhalofop, fenoxaprop, fluazifop, haloxyfop, quizalofopand mixtures thereof, as well as the isomers thereof, for example, fenoxaprop-P, fluazifop-P, haloxyfop-P, quizalofop-P; and a cyclohexanedione herbicide such as alloxydim, clethodim, sethoxydim, tepraloxydim and tralkoxydim, as well as salts or esters thereof), a protoporphyrinogen oxidase inhibiting herbicide (e.g. fomesafen), an enolpyruvate shikimate phosphate synthase inhibiting herbicide (e.g. glyphosate) and/or an auxin transport inhibitor such as semicarbazone (e.g. diflufenzopyr, in particular the sodium salt).

A particularly preferred mixture partner for compounds of formula (I) is glyphosate.

For the avoidance of doubt, even if not explicitly stated above, the mixing partners of the compound of formula (I) may also be in the form of any suitable agrochemically acceptable ester or salt, as mentioned e.g. in The Pesticide Manual, Thirteenth Edition, British Crop Protection Council, 2003.

The mixing ratio of the compound of formula (I) to the mixing partner is preferably from 1: 100 to 1000:1.

The mixtures can advantageously be employed in the above-mentioned formulations (in which case “active ingredient” relates to the respective mixture of compound of formula (I) with the mixing partner). Alternatively, each mixture partner may be formulated separately and subsequently combined, for example as a tank-mix.

Various aspects and embodiments of the present invention will now be illustrated in more detail by way of example. It will be appreciated that modification of detail may be made without departing from the scope of the invention.

For the avoidance of doubt, where a literary reference, patent application, or patent, is cited within the text of this application, the entire text of said citation is herein incorporated by reference.

### EXAMPLES

### Example 1

### Synthesis of 6-methoxycarbonyl-2,4,5-trichloropyrimidine

### Preparation of 2,4-dihydroxy-6-methoxycarbonylpyrimidine (methyl orotate)

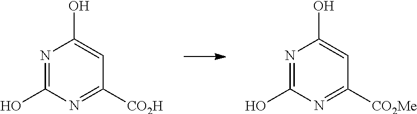

Thionyl chloride (500 ml), pyridine (2.5 ml) and a few drops of dimethylformamide were added to orotic acid monohydrate (78 g, 0.44 mol). The reaction mixture was stirred at RT for 5 days and then heated under reflux for an additional 14 hours. After cooling the solid material was allowed to settle and the supernatant decanted. The solid residue was washed with hexane and dried. Methanol (700 ml) was added dropwise with agitation to the solid. Once the rate of the gas formation slowed, the mixture was heated at reflux overnight and then cooled to 4-5° C. The solid was removed by filtration and washed with methanol and ether to provide methyl orotate (73 g, 97%).

¹H nmr (400 MHz, d₆-DMSO) δ_{H} 11.41 (1H, s), 11.26 (1H, s), 6.04 (1H, s), 3.84 (3H, s) ppm.

### 1.2 Preparation of 5-chloro-2,4-dihydroxy-6-methoxycarbonylpyrimidine

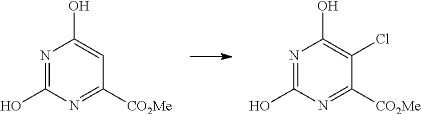

A catalytic quantity of ferric chloride was added to a solution of methyl orotate (34 g, 0.20 mol) in acetic anhydride (5% solution in glacial acetic acid, 500 ml). The mixture was heated to 90-95° C. and sulphuryl chloride (54 g, 0.40 mol) was added dropwise. After the addition was complete, the solution was slowly brought to reflux with agitation and heating was continued overnight. The solution was cooled to 18° C. and the solid was removed by filtration. The solid was washed with acetic acid and water, then dried to give 5-chloro-2,4-dihydroxy-6-methoxycarbonylpyrimidine (36 g, 89%).

¹H nmr (400 MHz, d₆-DMSO) δ_{H} 11.86 (1H, s), 11.62 (1H, s), 3.88 (3H, s) ppm.

### 1.3 Preparation of 6-methoxycarbonyl-2,4,5-trichloropyrimidine

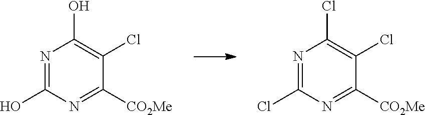

Phosphorus oxychloride (993 ml) was added to 5-chloro-2,4-dihydroxy-6-methoxycarbonylpyrimidine (30.0 g, 0.146 mol) at 10° C. and the resulting solution cooled to 0° C. N,N-Diethyl aniline (30.9 ml, 0.193 mol) was added dropwise to the stirred solution. After the addition was complete, the reaction mixture was allowed to warm slowly to ambient temperature and was then heated at reflux overnight. The resulting solution was cooled and concentrated under reduced pressure. The residue was poured onto crushed ice (600 g) and extracted with cold ether. The ether extracts were washed with brine, dried over sodium sulphate, filtered and evaporated under reduced pressure to give a light brown solid. This was triturated with warm hexane to yield 6-methoxycarbonyl-2,4,5-trichloropyrimidine (28 g, 82%).

¹H nmr (400 MHz, CDCl₃) δ_{H} 4.02 (3H, s) ppm.

### Example 2

### Synthesis of 4-amino-2,5-dichloro-6-methoxycarbonylpyrimidine

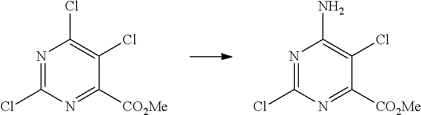

Aqueous ammonia (30% solution; 8.0 ml, 0.42 mol) was added dropwise to a stirred solution of 6-methoxycarbonyl-2,4,5-trichloropyrimidine (20.0 g, 0.083 mol) in THF (1000 ml) at 0° C. The reaction mixture was stirred at 0° C. for 1 hour and then filtered. The filtrate was evaporated under reduced pressure to give a white solid that was washed with twice with hexane and dried under vacuum to provide 4-amino-2,5-dichloro-6-methoxycarbonylpyrimidine (15.0 g, 82%).

¹H nmr (400 MHz, d₆-DMSO) δ_{H} 8.57 (1H, br s), 7.94 (1H, br s), 3.88 (3H, s) ppm.

### Example 3

### Synthesis of 4-amino-5-chloro-2-(4-chloro-3-dimethylamino-2-fluorophenyl)-6-methoxycarbonyl-pyrimidine.

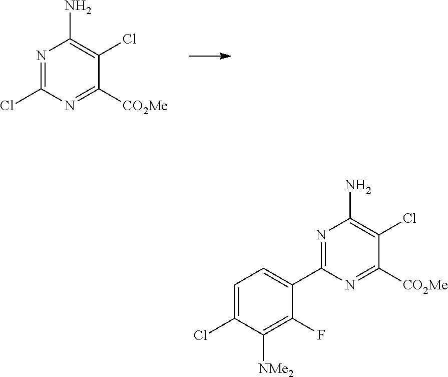

4-Chloro-3-dimethylamino-2-fluorophenyl boronic acid (1.08 g, 5.0 mmol), 4-amino-2,5-dichloro-6-methoxycarbonylpyrimidine (prepared as described in example 2) (1.2 g, 5.0 mmol), caesium fluoride (1.5 g, 10 mmol) and [1,1′-bis(diphenylphosphino)-ferrocene]dichloropalladium (II) complex with dichloromethane (1:1) (0.41 g, 0.50 mmol) were placed in a vial. Dimethoxyethane (6 ml) and water (6 ml) were added and the reaction mixture heated in a microwave reactor at 140° C. for 20 minutes, then allowed to cool, water added and the mixture extracted with dichloromethane. The organic extract was washed with brine, dried over magnesium sulphate, filtered and evaporated under reduced pressure. The residue was purified by chromatography on reverse phase silica using a gradient of methanol/water (3:2 to 4:1) as eluent to provide 4-amino-5-chloro-2-(4-chloro-3-dimethylamino-2-fluorophenyl)-6-methoxycarbonyl-pyrimidine as an off-white solid (780 mg, 45%).

¹H nmr (400 MHz, CDCl₃) δ_{H} 7.60 (1H, dd), 7.20 (1H, dd), 5.70 (2H, br s), 4.00 (3H, s), 2.90 (6H, s) ppm.

A further compound, prepared using this general method, is listed in Table 1 below.

TABLE 1
Compounds made according to the general method described in
Example 3 above.

| | | Melt- |
|---|---|---|
| | | ing |
| | | point |
| Name | Structure | (° C.) |
| 4-Amino-5-chloro-2- (4-chloro-2-fluoro-3- methoxyphenyl)-6- methoxycarbonyl- pyrimidine | 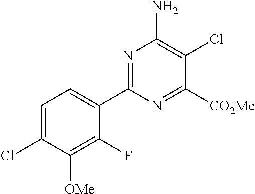 | 141-142 |

### Example 4

### Synthesis of 5-amino-2-(4-chloro-3-dimethylamino-2-fluorophenyl)-5-ethenyl-6-methoxycarbonylpyrimidine (Compound 1)

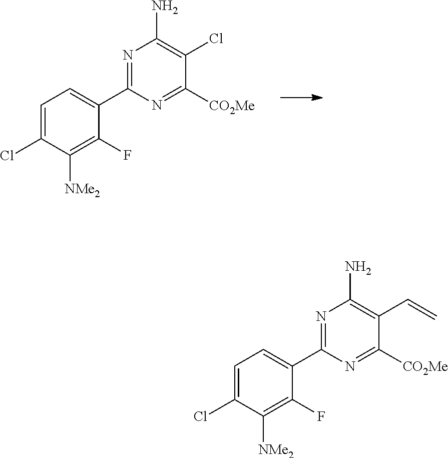

Vinyl boronic acid pinacol ester (86 μl, 0.51 mmol), 4-amino-5-chloro-2-(4-chloro-3-dimethylamino-2-fluorophenyl)-6-methoxycarbonyl-pyrimidine (prepared as described in example 3) (165 mg, 0.46 mmol), caesium fluoride (139 mg, 0.92 mmol) and [1,1′-bis(diphenylphosphino)-ferrocene]dichloropalladium (II) complex with dichloromethane (1:1) (38 mg, 46 μmol ) were placed in a vial. The vial was evacuated and backfilled with nitrogen before adding dimethoxyethane (2 ml) and water (2 ml). The reaction mixture was heated in a microwave reactor at 140° C. for 20 minutes, then allowed to cool and filtered through a plug of silica, washing with ethyl acetate. The filtrate was evaporated under reduced pressure and the crude product purified by chromatography on silica using a gradient of hexane/ethyl acetate (100:0 to 8:2) as eluent to provide 5-amino-2-(4-chloro-3-dimethylamino-2-fluorophenyl)-5-ethenyl-6-methoxycarbonylpyrimidine as a solid (68 mg, 42%).

M.p. 136-137° C.; ¹H nmr (400 MHz, CDCl₃) δ_{H} 7.60 (1H, t), 7.20 (1H, dd), 5.70 (2H, m), 5.40 (2H, br s), 3.90 (3H, s), 2.90 (6H, s) ppm.

Further compounds, prepared using this general method, are listed in Table 2 below.

TABLE 2
Compounds made according to the general method described in Example 4 above.

| Compound | | | Characteristic |
|---|---|---|---|
| No. | Name | Structure | data |
| 2 | 4-Amino-2-(4- chloro-2-fluoro- 3-methoxy- phenyl)- 5-ethenyl-6- methoxy- carbonyl- pyrimidine | 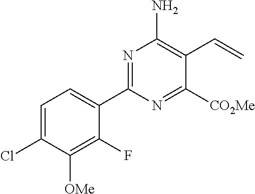 | 146-148 |
| 3 | (E)-4-Amino-2- (4-chloro-2- fluoro-3- methoxy- phenyl)-6- methoxy- carbonyl- 5-(prop-1- enyl)-pyrimidine | 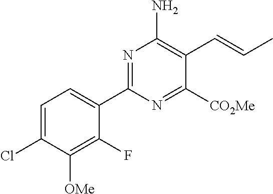 | 7.60 (1H, t), 7.20 (1H, d), 6.60 (1H, dd), 6.10 (1H, m), 5.30 (2H, br s), 4.00 (3H, s), 3.90 (3H, s), 1.90 (3H, d) |
| 4 | (Z)-4-Amino-2- (4-chloro-2- fluoro-3- methoxy- phenyl)-6- methoxy- carbonyl-5- (prop-1- enyl)-pyrimidine | 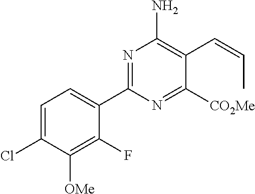 | 7.70 (1H, t), 7.30 (1H, dd), 6.40 (1H, dd), 6.10 (1H, m), 5.30 (2H, br s), 4.00 (3H, s), 3.90 (3H, s), 1.60 (3H, dd) |
| 5 | (Z)-4-Amino-2- (4-chloro-3- dimethylamino- 2-fluorophenyl)- 6- methoxy- carbonyl-5- (prop-1- enyl)-pyrimidine | 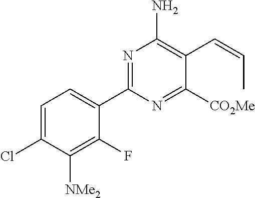 | 7.60 (1H, t), 7.20 (1H, d), 6.40 (1H, dd), 6.10 (1H, m), 5.40 (2H, br s), 3.90 (3H, s), 2.90 (6H, s), 1.60 (3H, dd) |
| 12 | (E)-4-Amino-2- (4-chloro-3- dimethylamino- 2-fluorophenyl)- 6-methoxy- carbonyl-5- (prop-1-enyl)- pyrimidine | 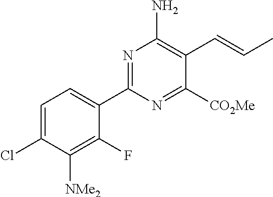 | 136-137 |

Characteristic data provided is either melting point(° C.) and/or ¹H-nmr data (400 MHz, CDCl₃) δ_{H} ppm

### Example 5

### Synthesis of 2,5-dichloro-4-(furan-2-ylmethylamino)-6-methoxycarbonylpyrimidine

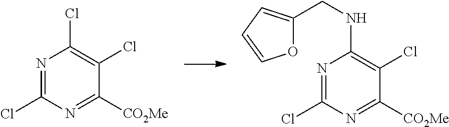

Furfurylamine (0.155 g, 1.60 mmol) was added to a stirred solution of 6-methoxycarbonyl-2,4,5-trichloropyrimidine (prepared as described in example 1) (0.193 g, 0.80 mmol) and triethylamine (0.24 ml, 1.7 mmol) in dichloromethane (3 ml). The solution was stirred at ambient temperature for 18 hours, and then added to a mixture of ethyl acetate and brine. The organic phase was dried over magnesium sulphate, filtered and evaporated under reduced pressure to give an orange solid. This was purified by column chromatography on silica using ethyl acetate:hexane (1:2) as eluent to provide 2,5-dichloro-4-(furan-2-ylmethylamino)-6-methoxycarbonylpyrimidine as a pale yellow solid (0.195 g, 81%).

M.p. 110-112° C.; ¹H nmr (400 MHz, CDCl₃) δ_{H} 7.40 (1H, m), 6.35 (2H, m), 6.16 (1H, br s), 4.72 (2H, d), 3.98 (3H, s) ppm.

Further compounds, prepared using this general method, are listed in Table 3 below.

TABLE 3
Compounds made according to the general method described in
Example 5 above.

| | | Characteristic |
|---|---|---|
| Name | Structure | Data |
| 4- Cyclopropylmethyl- amino-2,5-dichloro-6- methoxycarbonyl- pyrimidine | 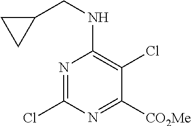 | 92-93 |
| 2,5-Dichloro-6- methoxycarbonyl-4- (2- nitrophenylmethyl- amino)-pyrimidine | 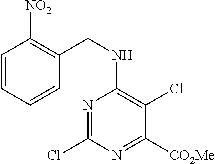 | 141-143 |
| 2,5-Dichloro-4-(2,4- dimethoxyphenyl- methylamino)-6- methoxycarbonyl- pyrimidine | 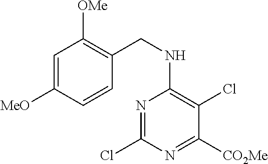 | 7.25 (1H, d), 6.47 (2H, m), 6.43 (1H, br t), 4.64 (2H, d), 3.96 (3H, s), 3.87 (3H, s), 3.82 (3H, s) |
| 2,5-Dichloro-6- methoxycarbonyl-4- (3-methylpyridin-2- ylmethylamino)- pyrimidine | 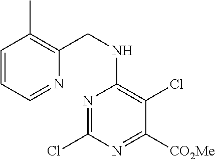 | 8.46 (1H, d), 8.38 (1H, br s), 7.54 (1H, d), 7.20 (1H, dd), 4.65 (2H, d), 3.99 (3H, s), 2.37 (3H, s) |
| 2,5-Dichloro-4-(4- fluorophenylmethyl- amino)-6- methoxycarbonyl- pyrimidine | 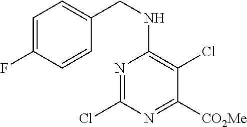 | 7.33 (2H, dd), 7.07 (2H, t), 6.11 (1H, br t), 4.71 (2H, d), 3.97 (3H, s), |
| 2,5-Dichloro-6- methoxycarbonyl-4- (1-phenyl- ethylamino)- pyrimidine | 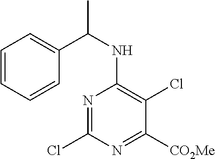 | 7.38 (4H, m), 7.32 (1H, m), 6.07 (1H, br d), 5.40 (1H, quintet), 3.96 (3H, s), 1.65 (3H, d) |

Characteristic data provided is either melting point (° C.) and/or ¹H-nmr data (400 MHz, CDCl₃) δ_{H} ppm

### Example 6

### Synthesis of 5-chloro-2-(4-chlorophenyl)-4-(furan-2-ylmethylamino)-6-methoxycarbonylpyrimidine

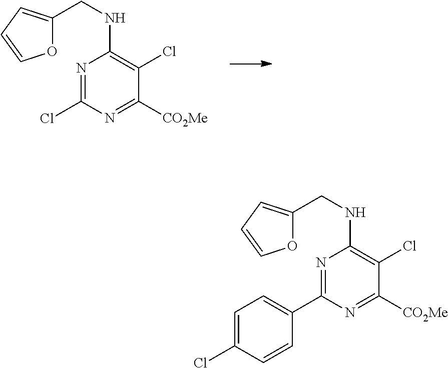

A solution of 4-chlorophenyl boronic acid pinacol ester (110 mg, 0.50 mmol), 2,5-dichloro-4-(furan-2-ylmethylamino)-6-methoxycarbonylpyrimidine (prepared as described in Example 5) (146 mg, 0.50 mmol), caesium fluoride (151 mg, 1.0 mmol) and [1,1′-bis(diphenylphosphino)-ferrocene]dichloropalladium (II) complex with dichloromethane (1:1) (41 mg, 0.05 mmol) in dimethoxyethane (1 ml) and water (1 ml) was heated in a microwave reactor at 140° C. for 10 minutes. The reaction mixture was allowed to cool, and then added to mixture of ethyl acetate (10 ml) and brine (10 ml). The organic phase was dried over magnesium sulphate, filtered and evaporated under reduced pressure to leave a brown gum. This was purified by chromatography on silica with 20% ethyl acetate in hexane as eluent to provide 5-chloro-2-(4-chlorophenyl)-4-(furan-2-ylmethylamino)-6-methoxycarbonylpyrimidine as a pale yellow solid (104 mg, 57%)_{.}

M.p. 132-134° C.; ¹H nmr (400 MHz, CDCl₃) δ_{H} 8.36 (2H, d), 7.42 (1H, m), 7.41 (2H, d), 6.36 (2H, m), 5.99 (1H, br t), 4.86 (2H, d), 4.03 (3H, s) ppm.

Further examples of compounds prepared using this method are listed below in Table 4.

TABLE 4
Compounds made according to the method described in Example 6
above.

| | | Characteristic |
|---|---|---|
| Name | Structure | Data |
| 5-Chloro-2-(4- chloro-2-fluoro-3- methoxyphenyl)-4- (furan-2- ylmethylamino)-6- methoxycarbonyl- pyrimidine | 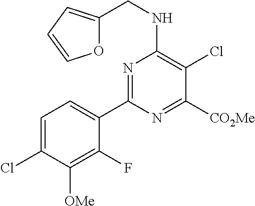 | 135-137 |
| 5-Chloro-2-(4- chloro-3- dimethylamino-2- fluorophenyl)-4- (furan-2- ylmethylamino)-6- methoxycarbonyl- pyrimidine | 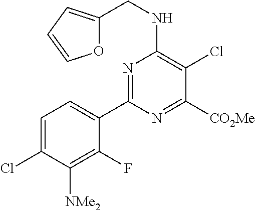 | 108-110 |
| 5-Chloro-2-(4- chloro-3- dimethylamino-2- fluorophenyl)-6- methoxycarbonyl- 4-(2- nitrophenylmethyl- amino)-pyrimidine | 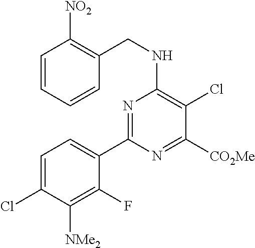 | 8.12 (1H, d), 7.76 (1H, d), 7.60 (2H, m), 7.49 (1H, t), 7.22 (1H, d), 6.70 (1H, t), 5.10 (2H, d), 4.00 (3H, s), 2.94 (6H, s) |
| 5-Chloro-2-(4- chloro-3- fluorophenyl)-6- methoxycarbonyl- 4-(3-methylpyridin- 2-ylmethylamino)- pyrimidine | 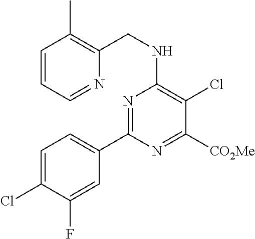 | 8.49 (1H, d), 8.22 (2H, m), 8.02 (1H, br t), 7.55 (1H, d), 7.48 (1H, dd), 7.21 (1H, m), 4.79 (2H, d), 4.04 (3H, s), 2.43 (3H, s) |
| 5-Chloro-2-(4- chloro-3- dimethylamino-2- fluorophenyl)-6- methoxycarbonyl- 4-(3-methylpyridin- 2-ylmethylamino)- pyrimidine | 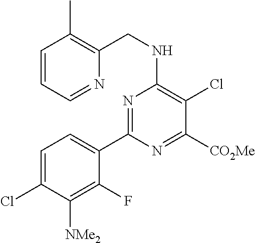 | 8.47 (1H, d), 7.99 (1H, br t), 7.66 (1H, t), 7.52 (1H, d), 7.24 (1H, m), 7.19 (1H, dd), 4.74 (2H, d), 4.01 (3H, s), 2.91 (6H, s), 2.37 (3H, s) |
| 5-Chloro-2-(4- chloro-3- dimethylamino-2- fluorophenyl)-4- (2,4- dimethoxyphenyl- methylamino)-6- methoxycarbonyl- pyrimidine | 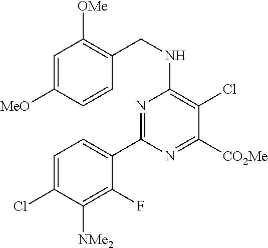 | 7.68 (1H, t), 7.28 (1H, d), 7.22 (1H, dd), 6.49 (1H, d), 6.44 (1H, dd), 6.29 (1H, br t), 4.72 (2H, d), 3.99 (3H, s), 3.88 (3H, s), 3.81 (3H, s), 2.92 (6H, s) |
| 5-Chloro-2-(4- chloro-3- dimethylamino-2- fluorophenyl)-4-(4- fluorophenylmethyl amino)-6- methoxycarbonyl- pyrimidine | 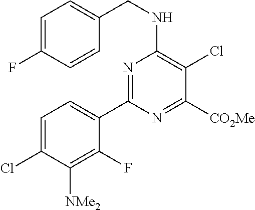 | 7.63 (1H, t), 7.36 (2H, m), 7.21 (1H, dd), 7.05 (2H, t), 6.05 (1H, br t), 4.78 (2H, d), 4.01 (3H, s), 2.91 (6H, s) |
| 5-Chloro-2-(4- chloro-2- fluorophenyl)-4- cyclopropylmethyl- amino-6- methoxycarbonyl- pyrimidine | 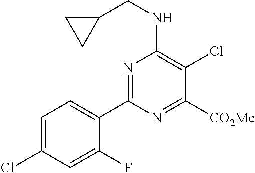 | 7.99 (1H, t), 7.20 (2H, m), 5.84 (1H, br t), 4.01 (3H, s), 3.46 (2H, m), 1.15 (1H, m), 0.61 (2H, m), 0.33 (2H, m) |
| 5-Chloro-4- cyclopropylmethyl- amino-2-(2,4- dichloro-3- fluorophenyl)-6- methoxycarbonyl- pyrimidine | 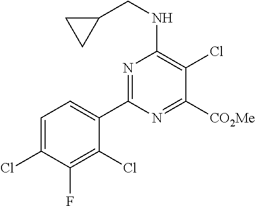 | 7.57 (1H, dd), 7.38 (1H, dd), 5.90 (1H, br t), 4.01 (3H, s), 3.44 (2H, m), 1.13 (1H, m), 0.62 (2H, m), 0.31 (2H, m) |
| 5-Chloro-2-(4- chloro-2- fluorophenyl)-6- methoxycarbonyl- 4-(1-phenyl- ethylamino)- pyrimidine | 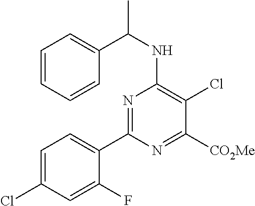 | 7.89 (1H, t), 7.39 (4H, m), 7.30 (1H, m), 7.16 (2H, m), 5.93 (1H, br d), 5.44 (1H, quintet), 3.99 (3H, s), 1.66 (3H, d) |

Characteristic data provided is either melting point (° C.) and/or ¹H-nmr data (400 MHz, CDCl₃) δ_{H} ppm

### Example 7

### Synthesis of (4-chloro-2-fluoro-3-methoxyphenyl)-4,5-dichloro-6-methoxycarbonyl-pyrimidine

### 7.1 Preparation of 3-bromo-6-chloro-2-fluorophenol

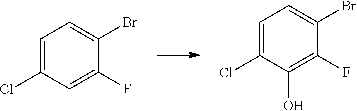

n-Butyllithium (2.1M in hexane; 68 ml, 149 mmol) was added dropwise to a stirred solution of diisopropylamine (15 g, 149 mmol) in anhydrous tetrahydrofuran (700 ml) at −78° C. under a nitrogen atmosphere. Once the addition was complete the reaction mixture was allowed to warm to 0° C., cooled to −78° C. and a solution of 1-bromo-4-chloro-2-fluorobenzene (25 g, 119 mmol) in anhydrous tetrahydrofuran (60 ml) was added dropwise. The mixture was allowed to warm to −20° C., cooled to −78° C. and a solution of trimethylborate (15 g, 143 mmol) in anhydrous tetrahydrofuran (30 ml) added dropwise. The reaction mixture was allowed to warm to −20° C. and stirred at that temperature for 30 minutes, then cooled to −78° C. and peracetic acid (80 ml) added dropwise. The mixture was allowed to warm to ambient temperature and stirred overnight under nitrogen. Water (1 l) was added and the resulting mixture extracted with ethyl acetate (3×500 ml). The combined organic extracts were washed with water and brine, dried over sodium sulphate, filtered and evaporated under reduced pressure. The residue was purified by column chromatography on silica using 2% ethyl acetate in hexane as eluent to provide 3-bromo-6-chloro-2-fluorophenol (13.8 g, 51%).

### 7.2 Preparation of 1-bromo-4-chloro-2-fluoro-3-methoxybenzene

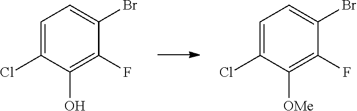

Anhydrous potassium carbonate (17 g, 122 mmol) was added to a solution of 3-bromo-6-chloro-2-fluorophenol (13.8 g, 61 mmol,) in anhydrous acetonitrile (100 ml). at ambient temperature. Methyl iodide (17 g, 122 mmol) was then added dropwise. The resulting mixture was heated at reflux for 2 hours, then cooled to ambient temperature and filtered through Celite®, the solid being washed with acetonitrile. The filtrate was evaporated under reduced pressure, the residue dissolved in ethyl acetate (250 ml) and washed with water (100 ml). The aqueous phase was extracted with ethyl acetate (100 ml and the combined organic phases were washed with brine, dried over sodium sulphate, filtered and evaporated under reduced pressure. The crude product was purified by column chromatography on silica using hexane as eluent to provide 1-bromo-4-chloro-2-fluoro-3-methoxybenzene as a colourless oil, which solidified on storage at 5° C. (11.4 g, 78%).

### 7.3 Preparation of 4-chloro-2-fluoro-3-methoxybenzonitrile

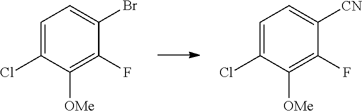

Copper(I) cyanide (57 g, 0.64 mol) was added to a degassed solution of 1-bromo-4-chloro-2-fluoro-3-methoxybenzene (76 g, 0.32 mol) in anhydrous dimethylformamide (760 ml). The resulting mixture was again degassed and tetrakis(triphenylphosphine)palladium(0) (1.2 g) added. The reaction mixture was heated at 110° C. under nitrogen for 24 hours, then allowed to cool to ambient temperature and water (2.5 l) added. The resulting mixture was stirred for 10 minutes, then filtered through Celite® and the solid washed with ethyl acetate (500 ml). The filtrate was separated into phases and the aqueous extracted with ethyl acetate (3×500 ml). The combined organic phases were washed with water and brine, dried over sodium sulphate, filtered and evaporated under reduced pressure. The crude product which was purified by column chromatography on silica using a gradient of ethyl acetate (2-5%) in hexane as eluent to provide 4-chloro-2-fluoro-3-methoxybenzonitrile as a colourless solid (38 g, 65%).

### 7.4 Preparation of 4-chloro-2-fluoro-3-methoxybenzamidine

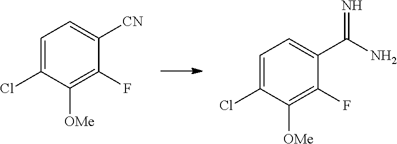

n-Butyllithium (2.2M in hexane; 196 ml, 0.43 mol) was added dropwise to a solution of hexamethyldisilazane (72 g, 0.45 mol) in anhydrous diethyl ether (800 ml) at −15° C. under a nitrogen atmosphere. The resulting solution was stirred at -15° C. for 1 hour, then a solution of 4-chloro-2-fluoro-3-methoxybenzonitrile (40 g, 0.22 mol) in anhydrous diethyl ether (600 ml) was added dropwise. The reaction mixture was stirred at −15° C. for 30 minutes, then allowed to warm to ambient temperature and stirring continued for 2 hours. The mixture was cooled to −10° C., hydrochloric acid (3M; 360 ml) added dropwise and the resulting mixture stirred at 0° C. for 45 minutes. The phases were separated and the aqueous layer washed with ethyl acetate (250 ml). The aqueous phase was cooled to 0° C., basified by the addition of aqueous sodium hydroxide (3M) and extracted with ethyl acetate (3×300 ml). The combined organic extracts were dried over sodium sulphate, filtered and evaporated under reduced to provide 4-chloro-2-fluoro-3-methoxybenzamidine as a yellow solid (33 g, 75%).

### 7.5 Preparation of 2-(4-chloro-2-fluoro-3-methoxyphenyl)-4-hydroxypyrimidine-6-carboxylic acid

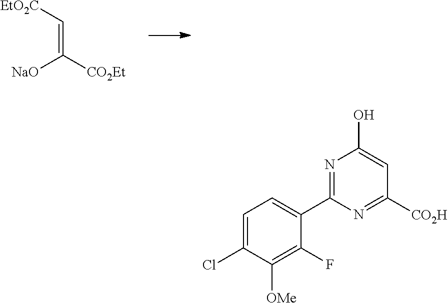

4-Chloro-2-fluoro-3-methoxybenzamidine (33.0 g, 160 mmol) was added to a stirred solution of diethyl oxaloacetate sodium salt (44.5 g, 200 mmol) in water (330 ml) and the resulting mixture heated at 70° C. for 24 hours, then cooled to ambient temperature. Water was added and the resulting solution basified by the addition of aqueous sodium hydroxide (10% solution). The mixture was extracted with ethyl acetate, then the aqueous phase cooled to 0° C. and acidified to pH2 by the cautious addition of hydrochloric acid (3N). The precipitate was isolated by filtration, washed with water and dried to yield 2-(4-chloro-2-fluoro-3-methoxyphenyl)-4-hydroxypyrimidine-6-carboxylic acid as a pale yellow solid (30 g, 60%).

### 7.6 Preparation of 5-chloro-2-(4-chloro-2-fluoro-3-methoxyphenyl)-4-hydroxypyrimidine-6-carboxylic acid

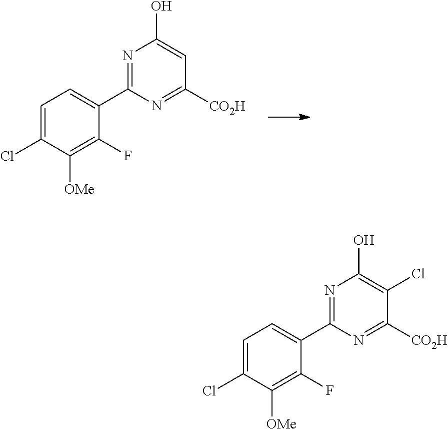

Aqueous sodium hypochlorite (10% solution; 287 ml) was added dropwise to a solution of 2-(4-chloro-2-fluoro-3-methoxyphenyl)-4-hydroxypyrimidine-6-carboxylic acid (30.0 g, 124 mmol) in concentrated hydrochloric acid (73 ml) and water (110 ml), maintaining the temperature below 15° C. during the course of the addition. The reaction mixture was stirred for 18 hours at ambient temperature. Further portions of aqueous sodium hypochlorite (10% solution; 287 ml) and concentrated hydrochloric acid (73 ml) were added at 24 hour intervals for 3 days. The reaction mixture was then filtered, the solid dissolved in saturated aqueous sodium bicarbonate and the solution washed with ethyl acetate. The aqueous phase was cooled to 0° C. and acidified to pH2 by the cautious addition of hydrochloric acid (3N). The precipitate was isolated by filtration, washed with water then dissolved in tetrahydrofuran. The solution was washed with water and the aqueous washings extracted with further tetrahydrofuran. The combined organic phases were dried over sodium sulphate, filtered and evaporated and the residue dried by azeotroping with toluene to provide 5-chloro-2-(4-chloro-2-fluoro-3-methoxyphenyl)-4-hydroxypyrimidine-6-carboxylic acid as an off-white solid (27 g, 80%).

### 7.7 Preparation of 2-(4-chloro-2-fluoro-3-methoxyphenyl)-4,5-dichloropyrimidine-6-carboxylic acid

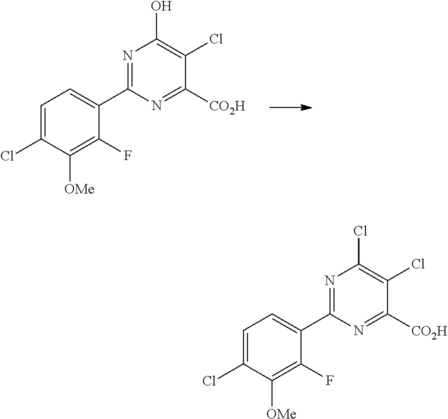

Dimethylformamide (1 drop) was added to a mixture of 5-chloro-2-(4-chloro-2-fluoro-3-methoxyphenyl)-4-hydroxypyrimidine-6-carboxylic acid (27.0 g, 810 mmol) and phosphorus oxychloride (108 ml) and the resulting mixture heated at 90° C. for 10 hours. The mixture was allowed to cool to ambient temperature and then added carefully to iced water and the resulting mixture extracted with ethyl acetate. The combined ethyl acetate layers were washed successively with water and brine, dried over sodium sulphate, filtered and the filtrate evaporated under reduced pressure to leave a residue that was triturated with hexane to provide 2-(4-chloro-2-fluoro-3-methoxyphenyl)-4,5-dichloropyrimidine-6-carboxylic acid as an off-white solid (24.2 g, 85%).

### 7.8 Preparation of 2-(4-chloro-2-fluoro-3-methoxyphenyl)-4,5-dichloro-6-methoxycarbonylpyrimidine

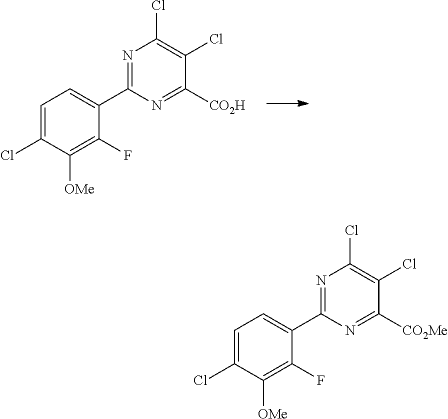

An excess of freshly prepared diazomethane was added to a solution of 2-(4-chloro-2-fluoro-3-methoxyphenyl)-4,5-dichloropyrimidine-6-carboxylic acid (12.5 g, 35.5 mmol) in methanol at -10° C. After stirring for 30 minutes the reaction mixture was concentrated under reduced pressure and the residue purified by column chromatography on silica using a gradient (0-2%) of ethyl acetate in hexane as eluent. The product was triturated with 1% ethyl in hexane to provide 2-(4-chloro-2-fluoro-3-methoxyphenyl)-4,5-dichloro-6-methoxycarbonylpyrimidine (9.5 g, 73%) as a white solid.

¹H nmr (400 MHz, CDCl₃) δ_{H} 7.78 (1H, dd), 7.26 (1H, dd), 4.05 (3H, s), 4.01 (3H, s)ppm.

### Example 8

### Synthesis of 5-chloro-2-(4-chloro-2-fluoro-3-methoxyphenyl)-6-methoxycarbonyl-4-(2-nitrophenylmethylamino)-pyrimidine

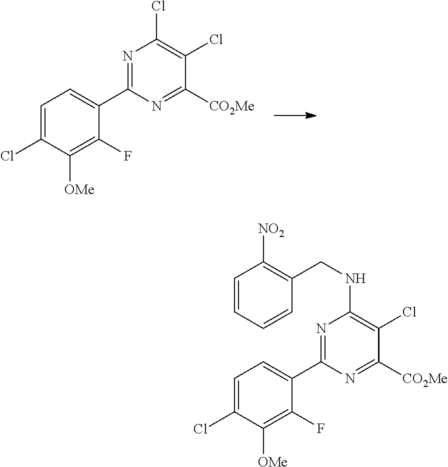

Triethylamine (3.7 ml, 26 mmol) was added to a stirred mixture of 2-(4-chloro-2-fluoro-3-methoxyphenyl)-4,5-dichloro-6-methoxycarbonylpyrimidine (prepared as described in example 7) (4.0 g, 11 mmol) and 2-nitrobenzylamine hydrochloride (3.1 g, 16 mmol), in dichloromethane (40 ml). The reaction mixture was stirred at ambient temperature for 5 hours, then ethyl acetate added and the resulting solution washed with brine, dried over magnesium sulphate, filtered and evaporated under reduced pressure. The residue purified by column chromatography on silica using dichloromethane as eluent to provide 5-chloro-2-(4-chloro-2-fluoro-3-methoxyphenyl)-6-methoxycarbonyl-4-(2-nitrophenylmethylamino)-pyrimidine as a yellow solid (4.45 g, 84%).

Characterising data for this compound are as follows:

Melting point: 123-125° C.;

¹H nmr (400 MHz, CDCl₃) δ_{H} 8.10 (1H, d), 7.73 (1H, d), 7.68 (1H, t), 7.61 (1H, t), 7.48 (1H, t), 7.28 (1H, d), 6.71 (1H, br t), 5.10 (2H, d), 3.98 (3H, s) ppm.

Further examples of compounds that were prepared using this method are listed below in Table 5. Characteristic data provided is either melting point (° C.) or ¹H-nmr data (400 MHz, CDCl₃) δ_{H}

TABLE 5
Compounds made according to the method described in Example 8 above.

| | | Characteristic |
|---|---|---|
| Name | Structure | data |
| 5-Chloro-2-(4- chloro-2-fluoro- 3-methoxyphenyl)- 4-(2-fluorophenyl- methylamino)-6- methoxycarbonyl- pyrimidine | 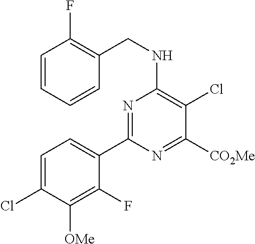 | 7.71 (1H, t), 7.41 (1H, t), 7.28 (1H, m), 7.21 (1H, d), 7.09 (2H, m), 6.16 (1H, t), 4.88 (2H, d), 4.00 (3H, s), 3.99 (3H, s) |
| 5-Chloro-2-(4- chloro-2-fluoro- 3-methoxyphenyl)- 4-cyclobutylamino- 6-methoxycarbonyl- pyrimidine | 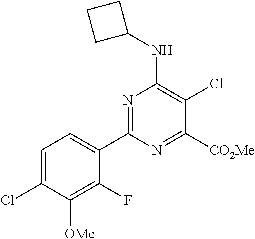 | 126-127 |

### Example 9

### Synthesis of 2-(4-chloro-2-fluoro-3-methoxyphenyl)-5-ethenyl-4-(furan-2-ylmethylamino)-6-methoxycarbonylpyrimidine (Compound 6)

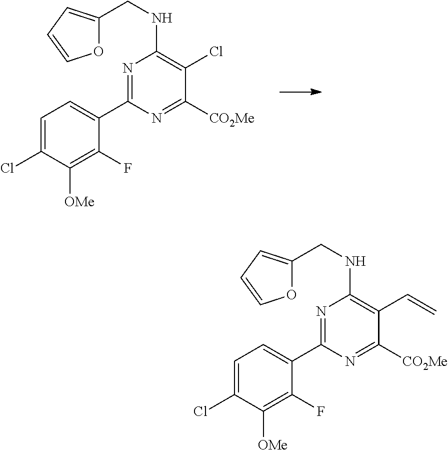

A solution of ethenyl boronic acid pinacol ester (360 mg, 2.4 mmol), 5-chloro-2-(4-chloro-2-fluoro-3-methoxyphenyl)-4-(furan-2-ylmethylamino)-6-methoxycarbonylpyrimidine (prepared as described in Example 6) (1.0 g, 2.4 mmol), caesium fluoride (710 mg, 4.7 mmol) and [1,1′-bis(diphenylphosphino)-ferrocene]dichloropalladium (II) complex with dichloromethane (1:1) (190 mg, 0.23 mmol) in dimethoxyethane (8 ml) and water (8 ml) was stirred for 1 minute, then heated in a microwave reactor at 150° C. for 20 minutes. The reaction mixture was allowed to cool, water added and the mixture extracted with dichloromethane. The organic extract was dried over magnesium sulphate, filtered and evaporated under reduced pressure. The residue was purified by chromatography on silica with a gradient of ethyl acetate in isohexane (0-50%) as eluent to provide 2-(4-chloro-2-fluoro-3-methoxyphenyl)-5-ethenyl-4-(furan-2-ylmethylamino)-6-methoxycarbonylpyrimidine as an off-white solid (620 mg, 63%).

M.p. 100-102° C.; ¹H nmr (400 MHz, CDCl₃) δ_{H} 7.76 (1H, t), 7.38 (1H, m), 7.22 (1H, m), 6.78 (1H, q), 6.34 (1H, m), 6.29 (1H, m), 5.83 (1H, br t), 5.68 (1H, d), 5.60 (1H, d), 4.75 (2H, d), 4.02 (3H, s), 3.93 (3H, s) ppm.

Further examples of compounds prepared using this method are listed below in Table 6.

TABLE 6
Compounds made according to the method described in Example 9
above.

| Compound | | | Characteristic |
|---|---|---|---|
| Number | Name | Structure | Data |
| 7 | 2-(4-Chlorophenyl)- 5-ethenyl-4-(furan- 2-ylmethylamino)- 6- methoxycarbonyl- pyrimi-dine | 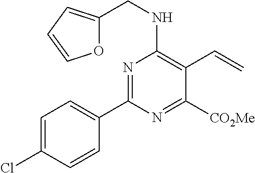 | 108-110 |
| 13 | 2-(4-Chloro-3- dimethylamino-2- fluorophenyl)-5- ethenyl-4-(furan-2- ylmethylamino)-6- methoxycarbonyl- pyrimi-dine | 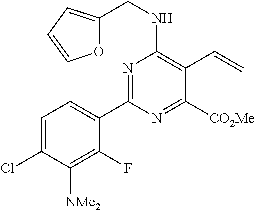 | 7.60 (1H, t), 7.38 (1H, m), 7.21 (1H, d), 6.78 (1H, m), 6.35 (1H, m), 6.28 (1H, m), 5.83 (1H, br t), 5.63 (2H, m), 4.75 (2H, d), 3.93 (3H, s), 2.92 (6H, s) |
| 14 | 2-(4-Chloro-2- fluoro-3- methoxyphenyl)-5- ethenyl-4-(2- fluorophenylmethyl amino)-6- methoxycarbonyl- pyrimi-dine | 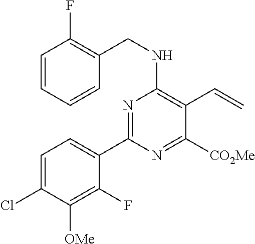 | 79-81 |
| 15 | 2-(4-Chloro-2- fluoro-3- methoxyphenyl)-5- ethenyl-6- methoxycarbonyl- 4-(2- nitrophenylmethyl- amino)-pyrimidine | 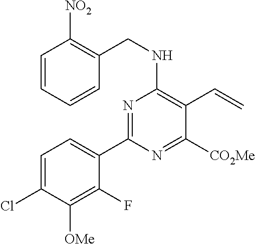 | 136-138 |
| 16 | 2-(4-Chloro-3- dimethylamino-2- fluorophenyl)-5- ethenyl-6- methoxycarbonyl- 4-(2- nitrophenylmethyl- amino)-pyrimidine | 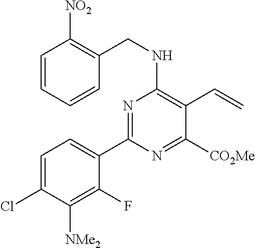 | 8.07 (1H, d), 7.77 (1H, m), 7.60 (2H, m), 7.46 (1H, m), 7.21 (1H, d), 6.77 (1H, d), 6.52 (1H, br t), 5.70 (1H, d), 5.58 (1H, d), 5.03 (2H, d), 3.93 (3H, s), 2.94 (6H, s) |
| 17 | 2-(4-Chloro-3- fluorophenyl)-5- ethenyl-6- methoxycarbonyl- 4-(3-methylpyridin- 2-ylmethylamino)- pyrimidine | 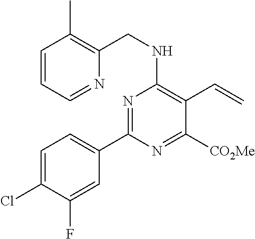 | 8.41 (1H, d), 8.07 (1H, t), 7.79 (1H, br t), 7.51 (1H, d), 7.21 (3H, m), 6.89 (1H, dd), 5.79 (2H, m), 4.72 (2H, d), 3.94 (3H, s), 2.36 (3H, s) |
| 18 | 2-(4-Chloro-3- dimethylamino-2- fluorophenyl)-5- ethenyl-6- methoxycarbonyl- 4-(3-methylpyridin- 2-ylmethylamino)- pyrimidine | 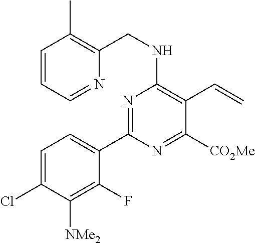 | 8.32 (1H, d), 7.59 (1H, d), 7.44 (1H, t), 7.21 (2H, m), 6.79 (1H, dd), 5.69 (2H, m), 4.76 (2H, s), 3.89 (3H, s), 2.87 (6H, s), 2.39 (3H, s) (NH not observed) [Solvent CD₃OD] |
| 19 | 2-(4-Chloro-3- dimethylamino-2- fluorophenyl)-4- (2,4- dimethoxyphenyl- methylamino)-5- ethenyl-6- methoxycarbonyl- pyrimidine | 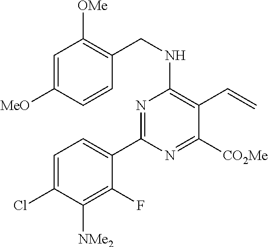 | 7.69 (1H, t), 7.26 (1H, d), 7.23 (1H, dd), 6.75 (1H, dd), 6.49 (1H, d), 6.43 (1H, dd), 6.19 (1H, t), 5.63 (1H, dd), 5.56 (1H, dd), 4.67 (2H, d), 3.91 (3H, s), 3.84 (3H, s), 3.79 (3H, s), 2.92 (6H, s) |
| 20 | 2-(4-Chloro-3- dimethylamino-2- fluorophenyl)-5- ethenyl-4-(4- fluorophenylmethyl amino)-6- methoxycarbonyl- pyrimi-dine | 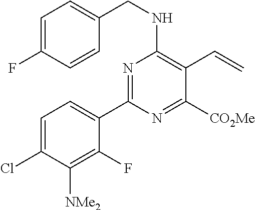 | 7.62 (1H, t), 7.22 (2H, m), 7.20 (1H, d), 7.02 (2H, t), 6.77 (1H, dd), 6.00 (1H, br t), 5.64 (1H, d), 5.59 (1H, d), 4.72 (2H, d), 3.91 (3H, s), 2.91 (6H, s) |
| 21 | 2-(4-Chloro-2- fluorophenyl)-4- cyclopropylmethyl- amino-5-ethenyl-6- methoxycarbonyl- pyrimidine | 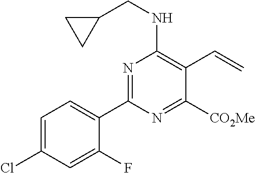 | 7.98 (1H, t), 7.13 (2H, m), 6.72 (1H, dd), 5.63 (2H, m), 5.56 (1H, d), 3.88 (3H, s), 3.35 (2H, dd), 1.06 (1H, m), 0.51 (2H, m), 0.24 (2H, m) |
| 22 | 4- Cyclopropylmethyl amino-2-(2,4- dichloro-3- fluorophenyl)-5- ethenyl-6- methoxycarbonyl- pyrimidine | 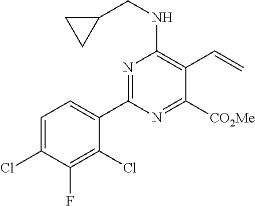 | 7.48 (1H, d), 7.32 (1H, m), 6.71 (1H, dd), 5.65 (1H, d), 5.59 (1H, d), 3.87 (3H, s), 3.56 (1H, br s), 3.31 (2H, d), 1.05 (1H, m), 0.49 (2H, m), 0.21 (2H, m) |
| 23 | 2-(4-Chloro-2- fluorophenyl)-5- ethenyl-6- methoxycarbonyl- 4-(1-phenyl- ethylamino)- pyrimidine | 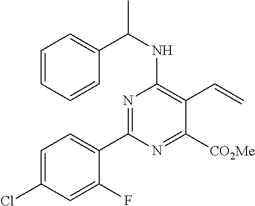 | 7.89 (1H, t), 7.38 (4H, m), 7.28 (1H, m), 7.16 (2H, d), 6.78 (1H, dd), 5.82 (1H, br d), 5.67 (1H, d), 5.61 (1H, d), 5.43 (1H, m), 3.91 (3H, s), 1.60 (3H, d) |
| 24 | 2-(4-Chloro-2- fluoro-3- methoxyphenyl)-4- cyclobutylamino-5- ethenyl-6- methoxycarbonyl pyrimidine | 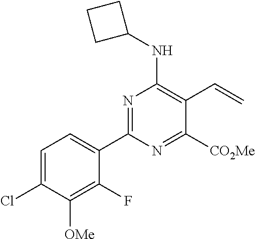 | 79-81 |

Characteristic data provided is either melting point (° C.) and/or ¹H-nmr data (400 MHz, CDCl₃) δ_{H} ppm

### Example 10

### Synthesis of 4-amino-2-(6-chloropyridin-3-yl)-5-ethenyl-6-methoxycarbonyl-pyrimidine (Compound 8)

### 10.1 Preparation of 4-amino-5-chloro-6-methoxycarbonyl-2-methylthiopyrimidine

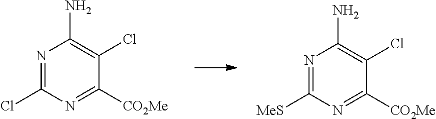

Sodium methanethiolate (3.0 g, 35 mmol) was added portionwise to a stirred solution of 4-amino-2,5-dichloro-6-methoxycarbonylpyrimidine (prepared as described in example 2) (4.4 g, 20 mmol) in methanol (100 ml) to give a pale yellow solution. The resulting mixture was stirred at reflux for 2 hours then allowed to cool for 2 hours, filtered and evaporated under reduced pressure. The residue was dissolved in water and ethyl acetate, the phases separated and the aqueous extracted with further ethyl acetate. The combined organic phases were washed with water and brine, dried over magnesium sulphate, filtered and evaporated under reduced pressure to provide 4-amino-5-chloro-6-methoxycarbonyl-2-methylthiopyrimidine as a yellow solid (2.2 g), which was used without further purification.

¹H nmr (400 MHz, CDCl₃) δ_{H} 5.55 (2H, br s), 3.95 (3H, s), 2.50 (3H, s) ppm.

### 10.2 Preparation of 4-amino-5-ethenyl-6-methoxycarbonyl-2-methylthiopyrimidine

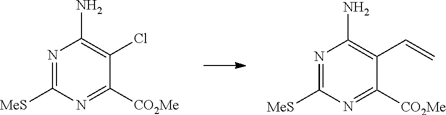

Water (2 ml) was added with stirring to a solution of 4-amino-5-chloro-6-methoxycarbonyl-2-methylthiopyrimidine (233 mg, 1.0 mmol) in dimethoxyethane (3 ml). The mixture was heated in a microwave reactor at 140° C. for 2 hours, then allowed to cool, diluted with ethyl acetate and washed with water and brine. The organic phase was dried over magnesium sulphate, filtered and evaporated under reduced pressure to provide a brown oil which was purified by chromatography on silica using hexane/ethyl acetate (4:1) as eluent to provide 4-amino-5-ethenyl-6-methoxycarbonyl-2-methylthiopyrimidine as a beige solid (120 mg, 50%).

¹H nmr (400 MHz, CDCl₃) δ_{H} 6.70 (1H, dd), 5.75 (2H, dd), 5.30 (2H, br s), 3.90 (3H, s), 2.50 (3H, s) ppm.

### 10.3 Preparation of 4-amino-2-(6-chloropyridin-3-yl)-5-ethenyl -6-methoxycarbonylpyrimidine

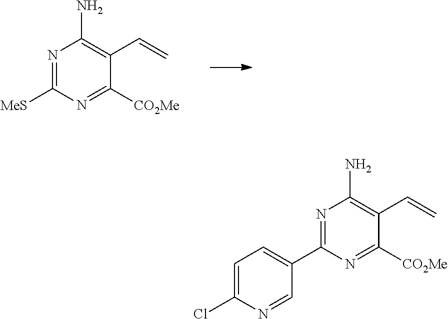

A solution of 4-amino-5-ethenyl-6-methoxycarbonyl-2-methylthiopyrimidine (113 mg, 0.50 mmol), 6-chloropyridine-3-boronic acid (85 mg, 0.55 mmol), copper thiophene-2-carboxylate (125 mg, 0.65 mmol), tri(2-furyl)phosphine (19 mg, 80 μmol) and tris(dibenzylideneacetone)dipalladium chloroform adduct (10 mg, 10 μmol) in tetrahydrofuran (3 ml) was heated in a microwave reactor at 100° C. for 30 minutes, then allowed to cool. Ether was added and the resulting solution washed with concentrated aqueous ammonia and brine, dried over magnesium sulphate, filtered and evaporated under reduced pressure to provide a yellow semi-solid (0.23 g). The crude product was purified by chromatography on silica using hexane/ethyl acetate (4:1) as eluent to provide 4-amino-2-(6-chloropyridin-3-yl)-5-ethenyl-6-methoxycarbonylpyrimidine as a white solid (40 mg, 27%).

M.p. 194-195° C.; ¹H nmr (400 MHz, CDCl₃) δ_{H} 9.32 (1H, s), 8.60 (1H, d), 7.40 (1H, d), 6.80 (1H, dd), 5.65 (2H, m), 5.45 (2H, br s), 3.97 (3H, s) ppm.

Further compounds, prepared using this general method, are listed in Table 7 below.

TABLE 7
Compounds made according to the general method described in
Example 10 above.

| Compound | | | Characteristic |
|---|---|---|---|
| No. | Name | Structure | Data |
| 9 | 4-Amino-2-(4- chloro-3- fluorophenyl)-5- ethenyl-6- methoxycarbonyl- pyrimidine | 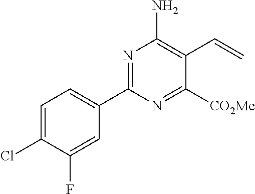 | 155-156 |
| 10 | 4-Amino-2-(4- chloro-2- fluorophenyl)-5- ethenyl-6- methoxycarbonyl- pyrimidine | 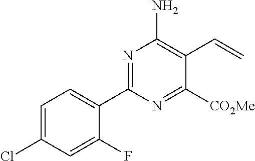 | 137-138 |
| 25 | 4-Amino-2-(3,4- dichlorophenyl)- 5-ethenyl-6- methoxycarbonyl- pyrimidine | 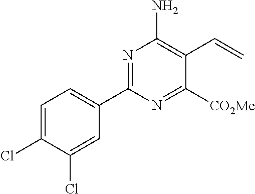 | 8.49 (1H, d), 8.22 (1H, dd), 7.50 (1H, d), 6.79 (1H, dd), 5.78 (1H, m), 5.64 (1H, dd), 5.40 (2H, br s), 3.98 (3H, s) |
| 26 | 4-Amino-2-(4- bromophenyl)- 5-ethenyl-6- methoxycarbonyl- pyrimidine | 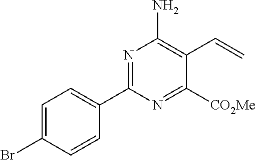 | 8.25 (2H, d), 7.56 (2H, d), 6.79 (1H, dd), 5.66 (1H, m), 5.63 (1H, dd), 5.38 (2H, br s), 3.96 (3H, s) |
| 27 | 4-Amino-2-(3- chloro-4- fluorophenyl)-5- ethenyl-6- methoxycarbonyl- pyrimidine | 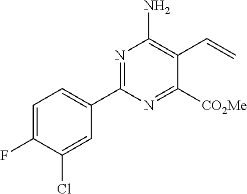 | 8.46 (1H, dd), 8.27 (1H, m), 7.19 (1H, t), 6.79 (1H, dd), 5.67 (1H, m), 5.63 (1H, dd), 5.39 (2H, br s), 3.96 (3H, s) |
| 28 | 4-Amino-5- ethenyl-2-(4- fluoro-3- methylphenyl)- 6- methoxycarbonyl- pyrimidine | 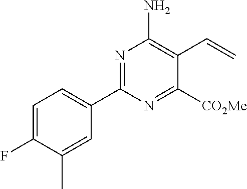 | 8.22 (1H, m), 8.18 (1H, m), 7.05 (1H, t), 6.79 (1H, dd), 5.66 (1H, m), 5.62 (1H, dd), 5.36 (2H, br s), 3.96 (3H, s), 2.34 (3H, s) |
| 29 | 4-Amino-2-(3,4- difluorophenyl)- 5-ethenyl-6- methoxycarbonyl- pyrimidine | 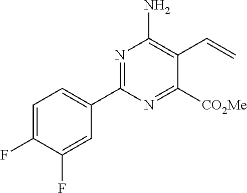 | 8.22 (1H, m), 8.15 (1H, m), 7.21 (1H, q), 6.80 (1H, dd), 5.67 (1H, d), 5.64 (1H, d), 5.38 (2H, br s), 3.97 (3H, s) |
| 30 | 4-Amino-2-(4- cyanophenyl)-5- ethenyl-6- methoxycarbonyl- pyrimidine | 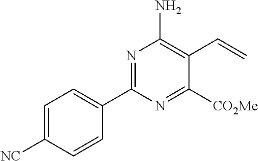 | 8.49 (2H, d), 7.74 (2H, d), 6.82 (1H, dd), 5.70 (1H, m), 5.66 (1H, dd), 5.41 (2H, br s), 3.97 (3H, s) |
| 31 | 4-Amino-5- ethenyl-6- methoxycarbonyl- 2-(4- trifluoromethoxy phenyl)- pyrimidine | 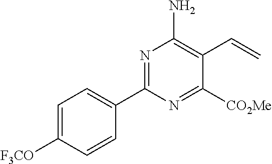 | 8.40 (2H, d), 7.27 (2H, d), 6.80 (1H, dd), 5.66 (1H, d), 5.63 (1H, d), 5.41 (2H, br s), 3.96 (3H, s) |
| 32 | 4-Amino-2-(4- chloro-3- methoxyphenyl)- 5-ethenyl-6- methoxycarbonyl- pyrimidine | 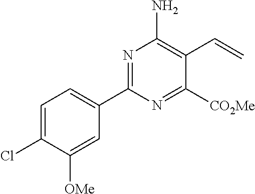 | 7.97 (1H, d), 7.95 (1H, dd), 7.42 (1H, d), 6.78 (1H, dd), 5.66 (1H, m), 5.63 (1H, dd), 5.39 (2H, br s), 4.03 (3H, s), 3.95 (3H, s) |
| 33 | 4-Amino-5- ethenyl-6- methoxycarbonyl- 2-(3,4- methylenedioxy phenyl)- pyrimidine | 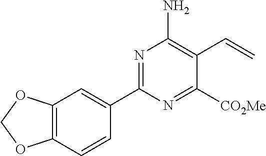 | 7.97 (1H, dd), 7.84 (1H, d), 6.88 (1H, d), 6.78 (1H, dd), 6.03 (2H, s), 5.84 (2H, br s), 5.67 (1H, dd), 5.63 (1H, dd), 3.96 (3H, s) |

Characteristic data provided is either melting point (° C.) and/or ¹H-nmr data (400 MHz, CDCl₃) δ_{H} ppm

### Example 11

### Synthesis of 4-amino-2-(4-chloro-2-fluoro-3-methoxyphenyl) -5-ethenylpyrimidine-6-carboxylic acid (Compound 11)

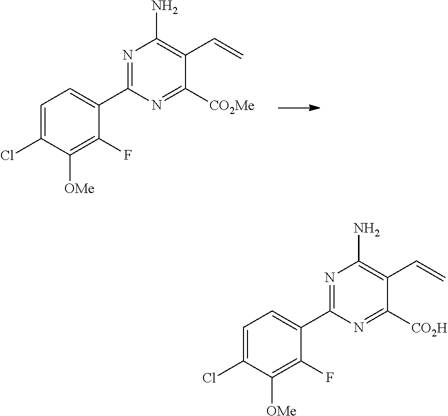

Sodium hydroxide (24 mg, 2 mmol) was added to a suspension of 4-amino-2-(4-chloro-2-fluoro-3-methoxyphenyl)-5-ethenyl-6-methoxycarbonylpyrimidine (prepared as described in example 4) (100 mg, 0.30 mmol) in tetrahydrofurn (10 ml) and water (6.5 ml) The reaction mixture was stirred for 3 hours at ambient temperature then acidified to pH 1-2 and washed with ethyl acetate. The aqueous phase was concentrated under reduced pressure to provide an off white solid which was purified using a FractionLynx automated hplc system to yield 4-amino-2-(4-chloro-2-fluoro-3-methoxyphenyl) -5-ethenylpyrimidine-6-carboxylic acid as a white solid (28 mg, 29%)

¹H nmr (400 MHz, d₆-DMSO) δ_{H} 7.50 (1H, m), 7.30 (1H, d), 6.50 (1H, m), 5.80 (1H, d), 5.30 (1H, d), 3.80 (3H, s) (amine and acid protons not observed).

Further compounds, prepared using this general method, are listed in Table 8 below.

TABLE 8
Compounds made according to the general method described in
Example 11 above.

| Compound | | | Characteristic |
|---|---|---|---|
| No. | Name | Structure | Data |
| 34 | 4-Amino-2-(4- chloro-3- dimethylamino- 2-fluorophenyl)- 5- ethenylpyrimidine- 6-carboxylic acid | 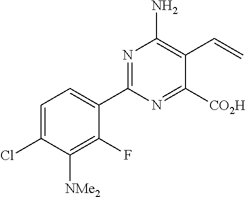 | 7.50 (1H, t), 7.20 (1H, t), 6.50 (3H, br s and t), 5.70 (1H, d), 5.20 (1H, d), 2.40 (6h, d) (acid proton not observed) |
| 35 | 2-(4-Chloro-2- fluoro-3- methoxyphenyl)- 5-ethenyl-4- (furan-2- ylmethylamino)- pyrimidine-6- carboxylic acid | 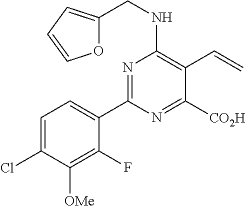 | 154-155 |

Characteristic data provided is either melting point (° C.) and/or ¹H-nmr data (400 MHz, CDCl₃) δ_{H} ppm

### Example 12

### Synthesis of 4-amino-2-(4-chloro-3-dimethylamino-2-fluorophenyl)-5-ethenyl-6-ethoxycarbonyl-pyrimidine (Compound 36)

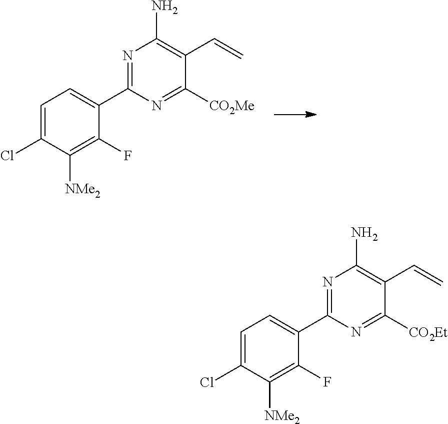

A solution of 4-amino-2-(4-chloro-3-dimethylamino-2-fluorophenyl)-5-ethenyl-6-methoxycarbonyl-pyrimidine (prepared by the method described in example 4) (200 mg, 0.57 mmol), 1-hydroxy-3-isothionato-1,1,3,3-tetrabutyl distannoxane (32 mg, 0.057 mmol), and ethanol (0.22 ml, 5.7 mmol) in toluene (8 ml) was heated at reflux for 3 hours. The reaction was allowed to cool and evaporated under reduced pressure and the residue purified by chromatography on silica using a hexane/ethyl acetate gradient (8:2 to 6:4) as eluent to provide 4-amino-2-(4-chloro-3-dimethylamino-2-fluorophenyl)-5-ethenyl-6-ethoxycarbonyl-pyrimidine as a white solid (193 mg, 93%).

¹H nmr (400 MHz, CDCl₃) δ_{H} 7.60 (1H, t), 7.20 (1H, dd), 6.80 (1H, dd), 5.70 (2H, dd), 5.50 (2H, br s), 4.40 (2H, q), 2.90 (6H, d), 1.40 (3H, t) ppm.

Further examples, prepared using this general method, are listed in Table 9 below.

TABLE 9
Compounds made according to the general method described in
Example 12 above.

| Compound | | | Characteristic |
|---|---|---|---|
| No. | Name | Structure | data |
| 37 | 4-Amino-2-(4- chloro-3- dimethylamino- 2-fluorophenyl)- 5-ethenyl-6- isopropoxy- carbonyl- pyrimidine | 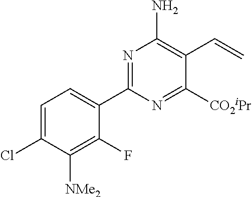 | 7.62 (1H, t), 7.21 (1H, dd), 6.72 (1H, dd), 5.67 (1H, m), 5.64 (1H, m), 5.36 (2H, br s), 5.28 (1H, septet), 2.90 (6H, d), 1.37 (6H, d) |
| 38 | 4-Amino-2-(4- chloro-3- dimethylamino- 2-fluorophenyl)- 5-ethenyl-6- ⁿpropoxy- carbonyl- pyrimidine | 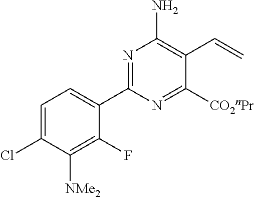 | 7.61 (1H, t), 7.22 (1H, dd), 6.76 (1H, dd), 5.68 (1H, s), 5.64 (1H, d), 5.42 (2H, br s), 4.31 (2H, t), 2.91 (6H, d), 1.80 (2H, sextet), 1.03 (3H, t) |
| 39 | 4-Amino-6- ⁿbutoxycarbonyl- 2-(4-chloro-3- dimethylamino- 2-fluorophenyl)- 5-ethenyl- pyrimidine | 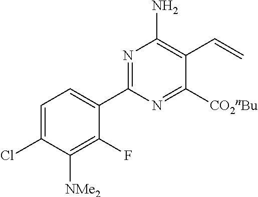 | 7.62 (1H, t), 7.22 (1H, dd), 6.77 (1H, dd), 5.68 (1H, s), 5.65 (1H, d), 5.39 (2H, br s), 4.36 (2H, t), 2.90 (6H, d), 1.76 (2H, m), 1.46 (2H, m), 0.96 (3H, t) |
| 40 | 4-Amino-2-(4- chloro-3- dimethylamino- 2-fluorophenyl)- 5-ethenyl-6-(2- methylpropoxy) carbonyl- pyrimidine | 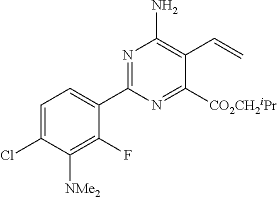 | 7.62 (1H, dd), 7.22 (1H, dd), 6.77 (1H, dd), 5.68 (1H, s), 5.64 (1H, d), 5.40 (2H, br s), 4.22 (1H, dd), 4.13 (1H, dd), 2.90 (6H, d), 1.93 (1H, m), 1.00 (3H, d), 0.92 (3H, t) |
| 41 | 4-Amino-2-(4- chloro-3- dimethylamino- 2-fluorophenyl)- 5-ethenyl-6- ⁿoctyloxycarbonyl- pyrimidine | 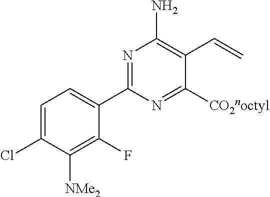 | 7.61 (1H, dd), 7.22 (1H, dd), 6.77 (1H, dd), 5.78 (1H, s), 5.64 (1H, d), 5.40 (2H, br s), 5.35 (2H, t), 2.90 (6H, d), 1.76 (2H, quintet), 1.43 (2H, m), 1.30 (8H, m), 0.88 (3H, m) |
| 42 | 4-Amino-2-(4- chloro-3- dimethylamino- 2-fluorophenyl)- 5-ethenyl-6- phenylmethoxy- carbonyl- pyrimidine | 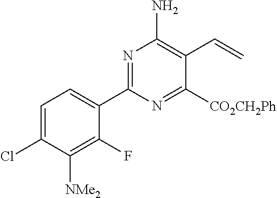 | 7.61 (1H, dd), 7.45 (2H, m), 7.36 (3H, m), 7.21 (1H, dd), 6.70 (1H, dd), 5.60 (1H, d), 5.56 (1H, s), 5.39 (2H, br s), 2.91 (6H, d) |
| 43 | 4-Amino-2-(4- chloro-3- dimethylamino- 2-fluorophenyl)- 5-ethenyl-6-(1- phenyl- ethoxy)carbonyl- pyrimidine | 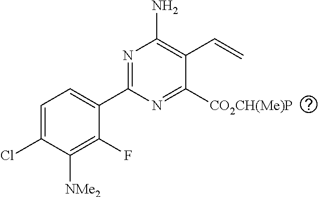 | 7.65 (1H, t), 7.45 (2H, m), 7.35 (3H, m), 7.22 (1H, dd), 6.64 (1H, dd), 6.14 (1H, q), 5.59 (1H, d), 5.53 (1H, dd), 5.38 (2H, br s), 2.91 (6H, d), 1.69 (3H, d) |
| 44 | 4-Amino-2-(4- chloro-3- dimethylamino- 2-fluorophenyl)- 5-ethenyl-6- (prop-2- enyloxy)carbonyl- pyrimidine | 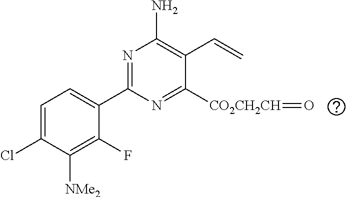 | 7.61 (1H, t), 7.22 (1H, dd), 6.78 (1H, dd), 6.02 (1H, m), 5.68 (1H, d), 5.65 (1H, dd), 5.47 (1H, m), 5.43 (2H, br s), 5.31 (1H, m), 4.85 (2H, m), 2.91 (6H, d) |
| 45 | 4-Amino-2-(4- chloro-3- dimethylamino- 2-fluorophenyl)- 5-ethenyl-6-(2- ethoxy- ethoxy)carbonyl- pyrimidine | 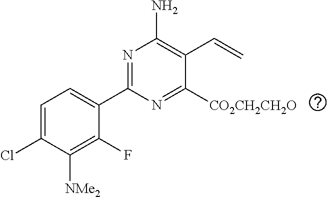 | 7.60 (1H, t), 7.22 (1H, dd), 6.79 (1H, dd), 5.69 (1H, d), 5.65 (1H, m), 5.41 (2H, br s), 4.50 (2H, m), 3.76 (2H, m), 3.58 (2H, q), 2.90 (6H, d), 1.25 (3H, t) |
| 46 | 4-Amino-6-(2- ⁿbutoxy- ethoxy)carbonyl- 2-(4-chloro-3- dimethylamino- 2-fluorophenyl)- 5-ethenyl- pyrimidine | 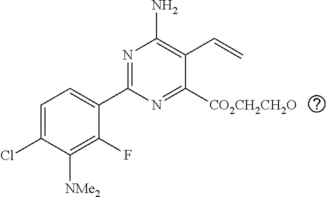 | 7.52 (1H, t), 7.24 (1H, dd), 6.77 (1H, dd), 5.75 (2H, m), 4.50 (2H, m), 3.75 (2H, m), 3.51 (2H, t), 3.29 (2H, br s), 2.91 (6H, d), 1.57 (2H, m), 1.38 (2H, m), 0.92 (3H, t) |
| 47 | 2-(4-Chloro-2- fluoro-3- methoxyphenyl)- 5-ethenyl-4- (furan-2- ylmethylamino)- 6- phenylmethoxy- carbonyl- pyrimidine | 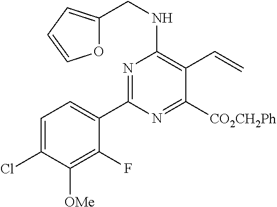 | 7.78 (1H, t), 7.44 (2H, m), 7.35 (3H, m), 7.35 (1H, m), 7.21 (1H, d), 6.65 (1H, m), 6.33 (1H, m), 6.28 (1H, m), 5.71 (1H, br t), 5.53 (2H, m), 5.38 (2H, s), 4.74 (2H, d), 4.01 (3H, s) |
| 48 | 2-(4-Chloro-2- fluoro-3- methoxyphenyl)- 5-ethenyl-6-(2- ethoxy- ethoxy)carbonyl- 4-(furan-2- ylmethylamino)- pyrimidine | 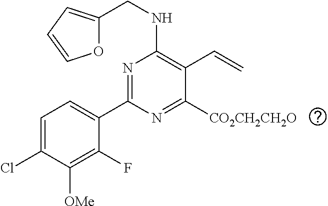 | 7.78 (1H, t), 7.47 (1H, m), 7.20 (1H, d), 6.72 (1H, m), 6.34 (1H, m), 6.28 (1H, m), 5.75 (1H, br t), 5.63 (2H, m), 4.77 (2H, d), 4.48 (2H, m), 4.00 (3H, s), 3.74 (2H, m), 3.57 (2H, q), 1.22 (3H, t) |

Characteristic data is ¹H nmr data (400 MHz, CDCl₃) δ_{H} ppm

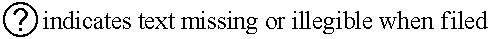

### Example 13

### Pre-Emergence Biological Efficacy

Seeds of Alopecurus myosuroides (ALOMY), Setaria faberi (SETFA), Echinochloa crus-galli (ECHCG), Solanum nigrum (SOLNI), Amaranthus retroflexus (AMARE) and Ipomea hederaceae (IPOHE) were sown in standard soil in pots. After cultivation for one day under controlled conditions in a glasshouse (at 24/16° C., day/night; 14 hours light; 65% humidity), the plants were sprayed with an aqueous spray solution derived from the formulation of the technical active ingredient in acetone/water (50:50) solution containing 0.5% Tween 20 (polyoxyethylene sorbitan monolaurate, CAS RN 9005-64-5) to give a final dose of 250 or 1000g/ha of test compound.

The test plants were then grown under controlled conditions in the glasshouse (at 24/16° C., day/night; 14 hours light; 65% humidity) and watered twice daily. After 13 days the test was evaluated (100 =total damage to plant; 0 =no damage to plant). Results are shown below in Table 10.

TABLE 10
Percentage damage caused to weed species by compounds
of the invention when applied pre-emergence.

| Compound | Dose | Species | | | | | |
|---|---|---|---|---|---|---|---|
| Number | (g/ha) | SOLNI | AMARE | IPOHE | SETFA | ALOMY | ECHCG |
| 1 | 1,000 | 100 | 100 | 90 | 90 | 80 | 90 |
| 2 | 1,000 | 100 | 100 | 90 | 90 | 90 | 90 |
| 3 | 1,000 | 80 | 50 | 90 | 80 | 90 | 90 |
| 4 | 1,000 | 100 | 100 | 100 | 80 | 80 | 90 |
| 5 | 1,000 | 60 | 70 | 20 | 30 | 30 | 30 |
| 6 | 1,000 | 100 | 100 | 90 | 80 | 70 | 80 |
| 7 | 1,000 | 20 | 100 | 40 | 10 | 50 | 20 |
| 8 | 1,000 | 40 | 100 | 40 | 10 | 10 | 0 |
| 9 | 1,000 | 100 | 100 | 100 | 30 | 50 | 70 |
| 10 | 1,000 | 100 | 100 | 100 | 70 | 80 | 70 |
| 11 | 1,000 | 100 | 100 | 100 | 100 | 90 | 100 |
| 12 | 1,000 | 80 | 100 | 0 | 80 | 70 | 90 |
| 13 | 1,000 | 90 | 90 | 20 | 60 | 70 | 90 |
| 14 | 1,000 | 60 | 20 | 50 | 0 | 0 | 0 |
| 15 | 1,000 | 100 | 100 | 100 | 0 | 50 | 70 |
| 16 | 1,000 | 30 | 50 | 0 | 70 | 60 | 70 |
| 24 | 1,000 | 80 | 100 | 80 | 0 | 10 | 50 |
| 25 | 1,000 | 70 | 90 | 10 | 0 | 0 | 30 |
| 26 | 1,000 | 90 | 100 | 80 | 30 | 70 | 50 |
| 27 | 1,000 | 40 | 50 | 10 | 30 | 20 | 40 |
| 28 | 1,000 | 40 | 100 | 0 | 50 | 50 | 40 |
| 29 | 1,000 | 70 | 100 | 50 | 30 | 30 | 30 |
| 30 | 250 | 30 | 10 | 0 | 0 | 0 | 0 |
| 31 | 1,000 | 0 | 0 | 0 | 0 | 0 | 0 |
| 32 | 1,000 | 80 | 100 | 20 | 0 | 0 | 0 |
| 33 | 1,000 | 100 | 100 | 80 | 70 | 80 | 90 |
| 34 | 1,000 | 100 | 100 | 80 | 100 | 90 | 100 |
| 35 | 1,000 | 100 | 100 | 100 | 90 | 70 | 90 |
| 36 | 1,000 | 90 | 100 | 30 | 90 | 60 | 90 |
| 37 | 1,000 | 80 | 40 | 0 | 20 | 20 | 20 |
| 38 | 1,000 | 80 | 90 | 10 | 70 | 60 | 90 |
| 39 | 1,000 | 100 | 100 | 20 | 90 | 70 | 100 |
| 40 | 1,000 | 60 | 60 | 10 | 20 | 20 | 40 |
| 41 | 1,000 | 80 | 100 | 0 | 90 | 70 | 100 |
| 42 | 1,000 | 80 | 100 | 20 | 80 | 70 | 90 |
| 43 | 1,000 | 40 | 40 | 0 | 30 | 20 | 30 |
| 44 | 1,000 | 100 | 100 | 30 | 80 | 60 | 90 |
| 45 | 1,000 | 90 | 100 | 50 | 80 | 70 | 90 |
| 46 | 1,000 | 100 | 100 | 50 | 90 | 80 | 100 |
| 47 | 1,000 | 80 | 70 | 80 | 50 | 30 | 90 |
| 48 | 1,000 | 0 | 0 | 0 | 0 | 0 | 0 |

### Example 14

### Post-Emergence Biological Efficacy

Seeds of Alopecurus myosuroides (ALOMY), Setaria faberi (SETFA), Echinochloa crus-galli (ECHCG), Solanum nigrum (SOLNI), Amaranthus retroflexus (AMARE) and Ipomea hederaceae (IPOHE) were sown in standard soil in pots. After cultivation for 8 days under controlled conditions in a glasshouse (at 24/16° C., day/night; 14 hours light; 65% humidity), the plants were sprayed with an aqueous spray solution derived from the formulation of the technical active ingredient in acetone/water (50:50) solution containing 0.5% Tween 20 (polyoxyethylene sorbitan monolaurate, CAS RN 9005-64-5) to give a final dose of 250 or 1000 g/ha of test compound.

The test plants were then grown on under controlled conditions in a glasshouse (at 24/16° C., day/night; 14 hours light; 65% humidity) and watered twice daily. After 13 days the test was evaluated (100=total damage to plant; 0=no damage to plant). Results are shown below in Table 11.

TABLE 11
Percentage damage caused to weed species by compounds
of the invention when applied post-emergence

| Compound | Dose | Species | | | | | |
|---|---|---|---|---|---|---|---|
| Number | (g/ha) | SOLNI | AMARE | IPOHE | SETFA | ALOMY | ECHCG |
| 1 | 1,000 | 100 | 100 | 90 | 100 | 90 | 100 |
| 2 | 1,000 | 80 | 100 | 80 | 100 | 90 | 90 |
| 3 | 1,000 | 100 | 100 | 90 | 100 | 90 | 100 |
| 4 | 1,000 | 80 | 100 | 100 | 90 | 70 | 80 |
| 5 | 1,000 | 80 | 100 | 30 | 70 | 60 | 70 |
| 6 | 1,000 | 90 | 100 | 80 | 90 | 80 | 80 |
| 7 | 1,000 | 90 | 100 | 80 | 0 | 30 | 40 |
| 8 | 1,000 | 90 | 100 | 50 | 0 | 0 | 0 |
| 9 | 1,000 | 100 | 100 | 100 | 40 | 40 | 60 |
| 10 | 1,000 | 100 | 100 | 80 | 70 | 90 | 70 |
| 11 | 1,000 | 90 | 100 | 90 | 100 | 90 | 90 |
| 12 | 1,000 | 80 | 100 | 40 | 80 | 80 | 80 |
| 13 | 1,000 | 100 | 100 | 40 | 80 | 70 | 80 |
| 14 | 1,000 | 90 | 100 | 60 | 0 | 0 | 0 |
| 15 | 1,000 | 90 | 100 | 90 | 70 | 70 | 90 |
| 16 | 1,000 | 80 | 100 | 60 | 90 | 70 | 70 |
| 24 | 1,000 | 90 | 100 | 70 | 10 | 10 | 40 |
| 25 | 1,000 | 90 | 100 | 70 | 0 | 20 | 20 |
| 26 | 1,000 | 90 | 100 | 70 | 30 | 60 | 50 |
| 27 | 1,000 | 90 | 100 | 30 | 60 | 40 | 50 |
| 28 | 1,000 | 70 | 100 | 30 | 50 | 50 | 40 |
| 29 | 1,000 | 90 | 100 | 60 | 10 | 20 | 40 |
| 30 | 250 | 80 | 30 | 40 | 0 | 0 | 0 |
| 31 | 1,000 | 70 | 100 | 10 | 10 | 10 | 10 |
| 32 | 1,000 | 80 | 100 | 70 | 0 | 50 | 60 |
| 33 | 1,000 | 90 | 100 | 80 | 40 | 100 | 100 |
| 34 | 1,000 | 90 | 100 | 70 | 100 | 90 | 100 |
| 35 | 1,000 | 90 | 100 | 70 | 80 | 70 | 80 |
| 36 | 1,000 | 90 | 100 | 60 | 80 | 80 | 90 |
| 37 | 1,000 | 100 | 100 | 30 | 70 | 40 | 70 |
| 38 | 1,000 | 90 | 100 | 40 | 90 | 70 | 100 |
| 39 | 1,000 | 80 | 100 | 80 | 90 | 70 | 90 |
| 40 | 1,000 | 90 | 100 | 50 | 40 | 20 | 80 |
| 41 | 1,000 | 90 | 100 | 80 | 60 | 70 | 80 |
| 42 | 1,000 | 90 | 100 | 40 | 90 | 90 | 90 |
| 43 | 1,000 | 80 | 100 | 30 | 50 | 30 | 60 |
| 44 | 1,000 | 100 | 100 | 30 | 100 | 70 | 100 |
| 45 | 1,000 | 90 | 100 | 60 | 90 | 70 | 100 |
| 46 | 1,000 | 90 | 100 | 80 | 90 | 80 | 90 |
| 47 | 1,000 | 80 | 100 | 80 | 80 | 60 | 80 |
| 48 | 1,000 | 50 | 40 | 60 | 0 | 0 | 0 |

### Example 15

### Post-Emergence Biological Efficacy

Seeds of crop and representative weed species were sown in standard soil in pots. After cultivation for 14 days under controlled conditions in a glasshouse (at 22/16° C., day/night; 16 hours light; 65% humidity), the plants were sprayed. The spray solution was prepared by dissolving the technical active ingredient in acetone containing 10.56 wt % Emulsogen EL, 42.22 wt % N-methylpyrrolidone and 2.22 wt % DPG-monoethyl ether to give a 5% stock solution. This was then diluted with water containing 0.2% (v/v) of the adjuvant X-77 to give the desired treatment concentration.

The test plants were then grown on under controlled conditions in a glasshouse (at 22/16° C., day/night; 16 hours light; 65% humidity) and watered twice daily. After 15 days the test was evaluated (100 =total damage to plant; 0 =no damage to plant). Results are shown below in Table 12.

TABLE 12
Percentage damage caused to soya and weed species by compounds of the invention and reference standards when applied post-emergence

| Cmpd | Dose | Species | | | | | | | | | | | | |
|---|---|---|---|---|---|---|---|---|---|---|---|---|---|---|
| No. | (g/ha) | GLXMA | EPHHL | SIDSP | ABUTH | XANST | IPOHE | AMARE | CHEAL | POLCO | KCHSC | SINAR | GALAP | VERPE |
| 1 | 30 | 0 | 90 | 60 | 90 | 100 | 40 | 100 | 90 | 90 | 80 | 10 | 20 | 80 |
| Ref | 30 | 50 | 80 | 80 | 90 | 90 | 70 | 100 | 80 | 90 | 80 | 70 | 30 | 70 |
| ex A | | | | | | | | | | | | | | |
| 2 | 8 | 60 | 90 | 80 | 80 | 90 | 90 | 80 | — | 70 | 30 | 10 | 80 | — |
| Ref | 8 | 90 | 100 | 70 | 90 | 100 | 80 | 100 | 80 | 80 | — | 80 | — | 80 |
| ex B | | | | | | | | | | | | | | |
| Ref | 8 | 70 | 30 | 40 | 30 | 0 | 70 | 10 | 60 | 0 | 0 | 0 | — | 50 |
| ex C | | | | | | | | | | | | | | |
| 3 | 125 | 0 | 90 | 80 | 80 | 80 | 80 | 100 | 90 | 90 | 80 | 70 | 70 | 80 |
| Ref | 125 | 80 | 80 | 60 | 50 | 60 | 70 | 60 | 60 | 20 | 10 | 80 | 80 | 10 [symbol] |
| ex D | | | | | | | | | | | | | | |
| 4 | 8 | 20 | 90 | 50 | 60 | 90 | 70 | 80 | — | 70 | 30 | 60 | 30 | 80 |
| 5 | 30 | 0 | 60 | 0 | 40 | 70 | 0 | 90 | 80 | 90 | 70 | 80 | — | 60 |
| 6 | 125 | 10 | 90 | 80 | 80 | 100 | 80 | 100 | 90 | 70 | 60 | 60 | 70 | 70 |
| Ref | 125 | 100 | 100 | 90 | 100 | 100 | 90 | 100 | 90 | 90 | 90 | 90 | 90 | 10 [symbol] |
| ex E | | | | | | | | | | | | | | |
| Ref | 125 | 80 | 80 | 60 | 60 | 60 | 70 | 40 | 80 | 60 | 30 | 80 | 80 | 0 |
| ex F | | | | | | | | | | | | | | |
| 7 | 30 | 0 | 0 | 0 | 70 | 50 | 70 | 50 | 20 | 40 | 40 | 60 | 60 | 80 |
| 8 | 250 | 10 | 40 | 20 | 60 | 80 | 50 | 100 | 100 | 60 | 60 | 70 | — | 80 |
| 9 | 125 | 0 | 80 | 80 | 80 | 100 | 80 | 100 | 90 | 70 | 90 | 80 | — | 80 |
| 10 | 15 | 0 | 50 | 50 | 90 | 70 | 70 | 50 | 80 | 70 | 30 | 80 | — | 10 [symbol] |
| 11 | 8 | 0 | 90 | 80 | 90 | 90 | — | 60 | 80 | 70 | 10 | 0 | — | 80 |

GLXMA Soybean
EPPHL Euphorbia heterophylla
SIDSP Sida spinosa
ABUTH Abutilon theophrasti
XANST Xanthium strumarium
IPOHE Ipomoea hederacea
AMARE Amaranthus retroflexus
CHEAL Chenopodium album
POLCO Polygonum convolvulus
KSHSC Kochia scoparia
SINAR Sinapis arvensis
GALAP Galium aparine
VERPE Veronica persica [symbol]
indicates data missing or illegible when filed

TABLE 13
Reference Standards

| Reference Example | Name | Structure |
|---|---|---|
| A | 4-Amino-5-chloro-2- (4-chloro-3- dimethylamino-2- fluorophenyl)-6- methoxycarbonyl- pyrimidine | 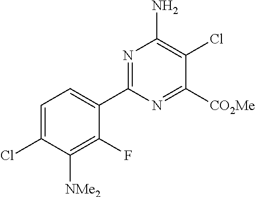 |
| B | 4-Amino-5-chloro-2- (4-chloro-2-fluoro-3- methoxyphenyl)-6- methoxycarbonyl- pyrimidine | 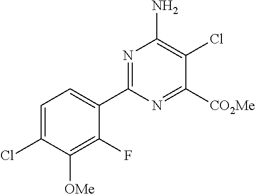 |
| C | 4-Amino-2- cyclopropyl-5-ethenyl- 6-methoxycarbonyl- pyrimidine | 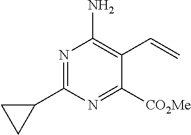 |
| D | (E)-4-Amino-2- cyclopropyl-6- methoxycarbonyl-5- (prop-1-enyl)- pyrimidine | 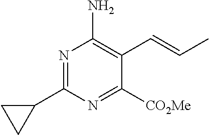 |
| E | 5-Chloro-2-(4-chloro- 2-fluoro-3- methoxyphenyl)-4- (furan-2- ylmethylamino)-6- methoxycarbonyl- pyrimidine | 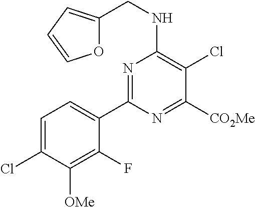 |
| F | 2-Cyclopropyl-5- ethenyl-4-(furan-2- ylmethylamino)-6- methoxycarbonyl- pyrimidine | 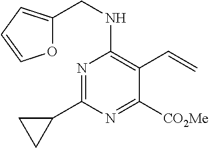 |
| G | 4-Amino-2-(4-chloro- 2-fluoro-3- methoxyphenyl)-6- methoxycarbonyl-5- methylpyrimidine | 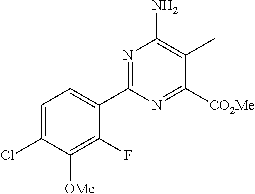 |
| H | (Z)-4-Amino-2- cyclopropyl-6- methoxycarbonyl-5- (prop-1-enyl)- pyrimidine | 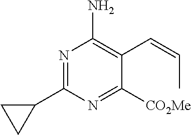 |
| J | 4-Amino-6-(4-chloro- 3-fluorophenyl)-3- ethenyl-2- methoxycarbonyl-5- methylpyridine | 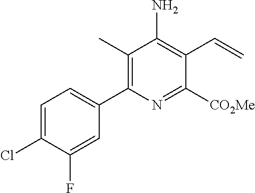 |

Notes:
These reference standards may be made, as the skilled man will appreciate, by applying and/or adapting as appropriate, the methods described in the prior art (see for example WO 2007/082076, WO 2009/046090, WO2009/081112).

### Example 16

### Pre-Emergence Biological Efficacy

Seeds of crop and representative weed species were sown in standard soil in pots. After cultivation for 1 day under controlled conditions in a glasshouse (at 22/16° C., day/night; 16 hours light; 65% humidity), the plants were sprayed. The spray solution was prepared by dissolving the technical active ingredient in acetone containing 10.56 wt % Emulsogen EL, 42.22 wt % N-methylpyrrolidone and 2.22 wt % DPG-monoethyl ether to give a 5% stock solution. This was then diluted with water containing 0.2% (v/v) of the adjuvant X-77 to give the desired treatment concentration.

The test plants were then grown on under controlled conditions in a glasshouse (at 22/16° C., day/night; 16 hours light; 65% humidity) and watered twice daily. After 20 days the test was evaluated (100=total damage to plant; 0=no damage to plant). Results are shown below in Table 14.

TABLE 14
Percentage damage caused to soya and weed species by compounds of the invention and reference standards when applied pre-emergence.

| Cmpd | Dose | Species | | | | | | | | | | | | |
|---|---|---|---|---|---|---|---|---|---|---|---|---|---|---|
| No. | (g/ha) | GLXMA | EPHHL | SIDSP | ABUTH | XANST | IPOHE | AMARE | CHEAL | POLCO | KCHSC | SINAR | GALAP | [symbol] |
| 2 | 15 | 20 | 100 | 80 | 40 | 0 | 30 | 40 | 70 | 20 | 100 | 30 | 100 | [symbol] |
| Ref | 15 | 90 | 80 | 40 | 30 | 0 | 0 | 70 | 30 | 0 | 0 | 80 | 70 | [symbol] |
| ex B | | | | | | | | | | | | | | |
| 3 | 30 | 0 | 100 | 90 | 30 | 10 | 0 | 80 | 100 | 50 | 100 | 40 | 100 | [symbol] |
| 4 | 125 | 10 | 100 | 100 | 100 | 30 | 50 | 100 | 40 | 20 | — | 50 | 80 | [symbol] |
| 5 | 125 | 0 | 100 | 90 | 70 | 40 | 50 | 90 | 100 | 0 | 30 | 40 | 100 | [symbol] |
| Ref | 125 | 10 | 100 | 70 | 40 | 20 | 30 | 70 | 30 | 0 | 60 | 70 | 90 | [symbol] |
| ex E | | | | | | | | | | | | | | |
| 11 | 250 | 0 | 100 | 100 | 100 | 70 | 100 | 100 | 100 | 20 | 100 | 70 | — | [symbol] |

[symbol]

indicates data missing or illegible when filed

### Species as for Post-Emergence Test.

The selectivity of compounds of formula (I) is clearly shown through the use of a Selectivity Index (SI), which is calculated as follows. Compounds are tested, using the methods described above, at 6 rates (typically from 8 g/ha to 250 g/ha) against a total of 17 species of broad leaf weeds and crops. Activity for the 17 species is averaged (mean) and an ED₅₀ value is calculated using standard techniques. The SI is then given by the following formula:

SI=log(ED₅₀ soybeans/ED₅₀[average 17 species])

Thus an SI of 0 indicates no selectivity, a negative SI indicates that the compound is more active against soybeans than against broad leaf plants in general and a positive SI indicates that a compound is less active against soybeans than broad leaf plants in general (i.e. selective). An SI of +0.5 equates to 3-fold selectivity, an SI of +1 to 10-fold selectivity and an SI of +2 to 100-fold selectivity.

Selectivity Indices for compounds of formula (I) and for reference examples are given below in Table 15.

TABLE 15
Selectivity Indices for compounds of the
invention and for reference examples

| | |
|---|---|
| Compound | Selectivity Index |

| Number | Pre-emergence | Post-emergence |
|---|---|---|
| 1 | +0.48 | +1.53 |
| 2 | +0.73 | — |
| 3 | +0.81 | +1.74 |
| 4 | +0.54 | +0.45 |
| 5 | +0.47 | +0.96 |
| 6 | +0.70 | +1.17 |
| 8 | — | +1.79 |
| 9 | — | +2.15 |
| 10 | — | +1.21 |
| 11 | >+0.65 | +1.10 |
| 13 | — | +2.20 |
| 14 | — | +1.70 |
| 15 | +0.47 | +0.97 |
| 16 | — | +2.14 |
| 17 | — | +0.56 |
| 18 | — | +1.97 |
| 19 | — | +1.61 |
| 20 | — | +0.46 |
| 21 | — | +0.64 |
| 22 | — | +1.35 |
| 23 | — | +1.68 |
| 24 | — | >+0.41 |
| 26 | +0.86 | +2.15 |
| 30 | — | +1.63 |
| 31 | — | +0.40 |
| 32 | — | +0.51 |
| 34 | +0.62 | +0.99 |
| 35 | +0.56 | +0.64 |
| 36 | — | +1.03 |
| 37 | — | +0.94 |
| 38 | — | +1.47 |
| 39 | — | +1.20 |
| 40 | +0.45 | +2.68 |
| 41 | +0.61 | +0.81 |
| 42 | — | +1.36 |
| 43 | — | +0.90 |
| 44 | +0.99 | +1.68 |
| 45 | — | +1.72 |
| 46 | +0.34 | +1.52 |
| 47 | — | +2.93 |
| Ref. Ex. A | +0.30 | +0.56 |
| Ref. Ex. B | −0.79 | −0.95 |
| Ref. Ex. C | −0.33 | −0.73 |
| Ref. Ex. D | +0.06 | −0.26 |
| Ref. Ex. E | — | −0.96 |
| Ref. Ex. F | +0.19 | −0.70 |
| Ref. Ex. G | −0.32 | +0.15 |
| Ref. Ex. H | 0 | −0.39 |
| Ref. Ex. J | −0.75 | −1.57 |

Thus it can be seen that compounds of formula (I) show good selectivity towards soybeans compared to other broad leaf plants. In comparison, the reference examples show little or no selectivity and in many cases are more injurious towards soybeans than to other broad leaf plants.

1. A method of controlling undesired plant growth in a crop of soya plants which comprises applying to said undesired plants or to the locus of said undesired plants, or to said soya plants, a compound of formula (I) 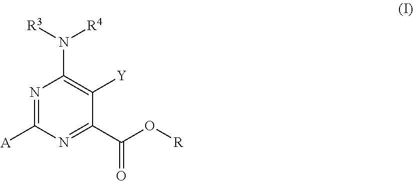
or salt or N-oxide thereof, wherein:
10 A is phenyl optionally substituted by 1-4 groups R¹, or pyridyl optionally substituted by 1-4 groups R¹; each R¹ is independently: halogen; cyano; nitro; alkyl; haloalkyl; alkoxyalkyl; alkoxy; haloalkoxy; alkylthio, alkylsulphinyl, alkylsulphonyl, alkylcarbonyl, alkoxycarbonyl; amino, alkylamino, dialkylamino; 15
R³ is hydrogen, C₁₋₄ alkyl, SO₂R⁶, or C(O)R⁷;
R⁴ is hydrogen, Ci₋₄ alkyl optionally substituted by 1-3 groups R⁵, C₃₋₆ cycloalkyl; 20. each R⁵ is independently: hydroxyl; cycloalkyl; phenyl optionally substituted by 1-3 groups R⁹; heteroaryl optionally substituted by 1-3 groups R¹⁰;
25 each R⁶ is independently C₁₋₄ alkyl or phenyl;
each R⁷ is independently C₁₋₄ alkyl, phenyl, or C₁₋₄ alkoxy;
each R⁹ is independently: halogen; cyano; nitro; alkyl; haloalkyl; alkoxy; 30 haloalkoxy; alkoxycarbonyl; amino; alkylamino; or dialkylamino;
each R¹⁰ is independently: halogen; cyano; alkyl; haloalkyl; alkoxy; haloalkoxy; alkoxycarbonyl; amino; alkylamino; or dialkylamino;
Y is C₂-₄alkenyl optionally substituted by 1-3 groups R¹⁴;
each R¹⁴ is independently halogen, cyano, cycloalkyl, alkylcarbonyl, alkoxycarbonyl, alkoxy, alkylthio, alkylsulphinyl, alkylsulphonyl; and,
R is hydrogen, C₁₋₁₀ alkyl, C₁₋₄alkoxyC₂-₄alkyl, C₃₋₁₀ alkenyl or phenylC₁₋₂ alkyl.

2. The method according to claim 1, wherein
A is phenyl optionally substituted by 1-3 groups R¹, or pyridyl optionally substituted by 1-3 groups R¹, each R¹ is independently: halogen; C₁₋₂alkyl; haloC₁₋₂alkyl; C₁̂alkOXyC₁₋₂alkyl; C₁₋₂alkoxy; haloC₁₋₂alkoxy; C₁₋₂alkylthio; amino, C₁₋₂alkylamino, di-C₁₋₂alkylamino;
R³ is hydrogen, C₁₋₂ alkyl;
R⁴ is hydrogen, C₁₋₂ alkyl optionally substituted by one or more R⁵; each R⁵ is independently: phenyl optionally substituted by 1-2 groups R⁹; furanyl optionally substituted by 1-2 groups R¹⁰; or pyridyl optionally substituted by 1-2 groups R¹⁰; each R⁹ is independently: halogen; cyano; nitro; C₁₋₂alkyl; C₁₋₂haloalkyl; C₁₋₂alkoxy; Ĉhaloalkoxy; amino; C₁₋₂alkylamino; or diĈalkylamino; each R¹⁰ is independently: halogen; cyano; C₁₋₂alkyl; C₁₋₂haloalkyl; C₁₋₂alkoxy; C₁₋₂haloalkoxy; amino; C₁₋₂alkylamino; or diC₁₋₂alkylamino; Y is C₂₋₄alkenyl or C₂₋₄haloalkenyl; and
R is hydrogen, C_{1-S} alkyl, C₁₋₄ alkoxyethyl, allyl or phenylmethyl.

3. The method according to claim 1, wherein A is 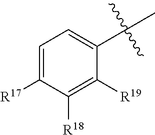
R¹⁷ is methyl, or halogen;
R¹⁸ is H, F, Cl, C₁₋₂alkyl, C₁₋₂haloalkyl, OR²⁰, or N(R²⁰)₂; R¹⁹ is H, F, or Cl; and each R²⁰ is independently H, C₁₋₂alkyl, C₁₋₂haloalkyl;
R⁴ is hydrogen, 2-nitrophenyl-methyl or furanylmethyl,
Y is C₂-₃ alkenyl; and, R is hydrogen, or Ci₋₄ alkyl.

4. The method according to claim 1, wherein said undesired plants are weeds selected from the following species: Euphorbia, Bidens, Ipomoea, Sida, Commelina, Conyza, Polygonum, Helianthus, Panicum, Eriochloa, Brachiaria, Cenchrus, Sorghum, and Scirpus.

5. The method according to claim 1, wherein the application of a compound of formula (I) is made post-emergence.

6. The method according to claim 1, wherein the application of a compound of formula (I) is made pre-emergence.

7. The method according to claim 1, wherein the compound of formula (I) is used in combination with at least one active ingredient selected from the group consisting of: an insecticide, an acaricide, a nematocide, a molluscicide, an herbicide, a fungicide, and a plant growth regulator.

8. The method according to claim 7, wherein the compound of formula (I) is used in combination with glyphosate.

9. The method according to any one claim 1 wherein the compound of formula (I) is formulated with at least one agriculturally acceptable formulation adjuvant or diluent.

10. Use of compound of formula (I) as defined in claim 1, to control undesired plant growth in soya crops.

11. A compound of formula (I): 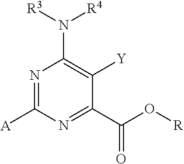
wherein:
A is 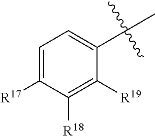
R¹⁷ is methyl, or halogen;
R¹⁸ is H, F, Cl, Ĉalkyl, C₁₋₂haloalkyl, or N(R²⁰)₂;
R¹⁹ is H, F, or Cl; and each R²⁰ is independently H, Ci₋₂alkyl, C₁₋₂haloalkyl; provided that both R¹⁸ and R¹⁹ are not hydrogen.
R³ is hydrogen, Ĉalkyl, SO₂R⁶, or C(O)R⁷;
R⁴ is C₁₋₄alkyl substituted by 1-3 groups R⁵, C₃₋₆ cycloalkyl;
each R⁵ is independently: hydroxyl; cycloalkyl; phenyl optionally substituted by 1-3 groups R⁹; heteroaryl optionally substituted by 1-3 groups R¹⁰;
each R⁶ is independently C₁₋₄alkyl or phenyl;
each R⁷ is independently C₁₋₄alkyl, phenyl, or Ci₋₄alkoxy;
each R⁹ is independently: halogen; cyano; nitro; alkyl; haloalkyl; alkoxy; haloalkoxy; alkoxycarbonyl; amino; alkylamino; or dialkylamino;
each R¹⁰ is independently: halogen; cyano; alkyl; haloalkyl; alkoxy; haloalkoxy; alkoxycarbonyl; amino; alkylamino; or dialkylamino;
Y is C₂₋₄alkenyl optionally substituted by 1-3 groups R¹⁴;
each R¹⁴ is independently halogen, cyano, cycloalkyl, alkylcarbonyl, alkoxycarbonyl, alkoxy, alkylthio, alkylsulphinyl, alkylsulphonyl; and,
R is hydrogen, C₁₋₁₀ alkyl, C₁₋₄alkoxyC₂₋₄alkyl, C₃₋₁₀ alkenyl or phenylCi₋₂ alkyl.
